(19) United States
(12) Patent Application Publication
(10) Pub. No.: US 2026/0097098 A1
(43) Pub. Date: Apr. 9, 2026

(54) Methods and Compositions for Treatment of Angiogenic Disorders Using Anti-VEGF Agents

(71) Applicant: The Regents of the University of California, Oakland, CA (US)

(72) Inventor: Napoleone Ferrara, La Jolla, CA (US)

(21) Appl. No.: 19/410,663

(22) Filed: Dec. 5, 2025

Related U.S. Application Data
(63) Continuation of application No. 18/640,840, filed on Apr. 19, 2024, now Pat. No. 12,514,905.
Application No. 18/640,840 is a continuation of application No. 18/053,311, filed on Nov. 7, 2022, now abandoned.
Application No. 18/053,311 is a continuation of application No. 16/962,529, now Pat. No. 11,524,053.
(60) Provisional application No. 62/622,382, filed on Jan. 26, 2018.

(51) Int. Cl. A61K 38/17; A61K 9/00

(52) U.S. Cl. CPC A61K 38/179; A61K 9/0019; A61K 9/0048

(57) ABSTRACT

Provided are methods and compositions for treatment of angiogenic disorders using anti-VEGF agents. The anti-VEGF agents comprise VEGF binding domains and have the ability to bind vitreous. Provided are exemplary embodiments of Fc-IgG fusion proteins with VEGF binding domains with strong heparin-binding characteristics, strong inhibition of VEGF mitogenic activity, and improved pharmacokinetics, namely longer half-lives of the anti-VEGF agents and consequently less frequent dosing.

### CROSS REFERENCE TO RELATED APPLICATIONS

This application is a continuation of U.S. application Ser. No. 18/640,840, filed Apr. 19, 2024, which is a continuation of U.S. application Ser. No. 18/053,311, filed Nov. 7, 2022, now abandoned, which is a continuation application of U.S. application Ser. No. 16/962,529, filed Jul. 16, 2020, now U.S. Pat. No. 11,524,053, which is a § 371 U.S. National Stage application of PCT Application No. PCT/US2019/015160, filed Jan. 25, 2019, which claims the priority benefit of U.S. Provisional Application No. 62/622,382, filed Jan. 26, 2018, the entire contents of which are incorporated herein by reference.

### SEQUENCE LISTING

The contents of the electronic sequence listing (sequencelisting.xml; Size: 34,000 bytes; and Date of Creation: Nov. 7, 2022) is herein incorporated by reference in its entirety.

### BACKGROUND

The development of a neovascular supply or angiogenesis serves crucial homeostatic roles since the blood vessels carry nutrients to tissues and organs and remove catabolic products¹. However, uncontrolled growth of blood vessels can promote or facilitate numerous disease processes, including tumors and intraocular vascular disorders¹. Although several angiogenic factors were initially identified and characterized (e.g., EGF, TGF-α, TGF-β, aFGF, bFGF, angiogenin)², work conducted over the last three decades has established the critical role of VEGF-A (VEGF hereafter) in normal and pathological angiogenesis ^{3 4}. VEGF is a member of a gene family that also includes PlGF, VEGF-B, VEGF-C and VEGF-D. Three related receptor tyrosine kinase (RTKs) have been reported to bind VEGF ligands: VEGFR-1, VEGFR-2 and VEGFR-3 ⁵. PlGF and VEGF-B interact selectively with VEGFR-1, VEGF binds both VEGFR-1 and VEGFR-2. A third member of this family of RTKs, VEGFR-3 ⁶, binds VEGF-C and VEGF-D, which are implicated in lymphangiogenesis. Each member of this RTK class has seven immunoglobulin (Ig)-like domains in the extracellular portion ⁷. There is agreement that VEGFR-2 is the main signaling receptor for VEGF⁵. However, VEGFR-1 binds VEGF with substantially higher binding affinity than VEGFR-2⁷.

VEGF inhibitors have become a standard of therapy in multiple tumors and have revolutionized the treatment of intraocular neovascular disorders such as the neovascular form of age-related macular degeneration (AMD), proliferative diabetic retinopathy and retinal vein occlusion, which are leading causes of severe vision loss and legal blindness ^{8 4}. Currently, three anti-VEGF drugs are widely used in the USA for ophthalmological indications: bevacizumab, ranibizumab and aflibercept⁴. Bevacizumab is a full-length IgG antibody targeting VEGF⁹. Even though bevacizumab was not developed for ophthalmological indications, it is widely used off-label due to its low cost. Ranibizumab is an affinity-matured anti-VEGF Fab¹⁰. Aflibercept is an IgG-Fc fusion protein^{11,12}, with elements from VEGFR-1 and VEGFR-2, that binds VEGF, PIGF and VEGF-B¹³. Conbercept is a soluble VEGF receptor structurally related to aflibercept, widely used as treatment of intraocular neovascularization in China¹⁴. Millions of patients worldwide have been treated with these drugs. Importantly, after five-year treatment with ranibizumab or bevacizumab, about half of neovascular AMD patients had good vision, i.e. visual acuity 20/40 or better, an outcome that would have been out of reach before anti-VEGF agents were available¹⁵.

However, in real-life clinical settings, many patients receive fewer anti-VEGF injections than in clinical trials and it has been hypothesized that this may correlate with poor visual outcomes ¹⁶. Indeed, the need to perform relatively frequent intravitreal injections has hampered patient compliance and ultimately the benefits of the therapy, especially in some countries¹⁶. Therefore, there is a need to develop agents with longer duration when injected in the eye, thus reducing the frequency of injections and a number of approaches to this end have been attempted ^{17, 18}. Aflibercept (EYLEA®) was approved based on clinical trials showing that every 8-week administration of the dose of 2 mg could match the efficacy of monthly ranibizumab (0.5 mg). However, despite the prediction that a switch to aflibercept would reduce the number of intravitreal injections, recent studies suggest that it is not the case ¹⁹. Therefore, there is still an unmet medical need for intravitreal anti-VEGF agents with improved half-life.

In 1996, Davis-Smyth et al. ²⁰ (see also U.S. Pat. No. 5,952,199) reported that domain (D) 2 of VEGFR-1 is the critical binding element for VEGF and PIGF. Deletion of D2 completely abolished binding. Replacing D2 of VEGFR-3 with VEGFR-1 D2 conferred on VEGFR-3 the ligand specificity of VEGFR-1²⁰. Subsequent work documented the interaction between D2 and VEGF (or PIGF) by X-ray crystallography ²¹⁻²³.

The initial studies led to the design of a construct with full VEGF binding characteristics, comprising the first three Ig-like Ds of VEGFR-1, fused to an Fc-IgG (Flt-1-3-IgG) ²⁰. Flt-1-3-IgG showed a potent ability to neutralize VEGF, in vitro and in vivo ²⁴⁻²⁷. However, the systemic half-life of this molecule was hampered by the presence of D3, which has significant heparin affinity due to the presence of clusters of basic residues, resulting in binding to HSPGs in various tissues. In 2002 Holash et al. ¹³ (U.S. Pat. No. 7,070,959) described an IgG fusion construct comprising of VEGFR-1 D2 (the binding element) and D3 of VEGFR2, which has much weaker heparin affinity compared to VEGFR-1 D3. This molecule, known today as aflibercept (marketed as EYLEA®), was reported to have a longer half-life compared to Flt-(1-3-IgG) following systemic administration¹³, clearly an advantage for treatment aiming, for example, at oncological indications.

### SUMMARY OF THE INVENTION

The present invention provides compositions and methods for inhibiting angiogenesis and for treating VEGF-associated conditions, such as ocular disease, including but not limited to, age-related macular degeneration, proliferative diabetic retinopathy, retinal vein occlusion, choroidal neovascularization secondary to myopia, retinopathy of prematurity, diabetic macular edema, polypoidal choroidal vasculopathy, comprising administering an anti-VEGF agent that inhibits the activity of VEGF and, at the same time, has strong heparin-binding characteristics, thereby providing superior pharmacokinetics, namely having a longer half-life of the therapeutic agent following intravitreal administration.

In embodiments, the present invention provides compositions and methods of treating conditions in which local direct administration of an anti-VEGF agent is beneficial, for example, treating and preventing endothelial cell proliferative conditions or angiogenesis, for example, in treating solid tumors, such as but not limited to, intracranial administration in glioblastomas.

In embodiments, the present invention provides an anti-VEGF agent, wherein the anti-VEGF agent is an Fc-IgG construct fusing domains with VEGF binding characteristics and domains that bind heparin proteoglycans. In embodiments, the present invention provides an anti-VEGF agent, wherein the anti-VEGF agent is an Fc-IgG construct having the ability to bind heparin and contains one or more domains with VEGF binding characteristics. In embodiments, the present invention provides an anti-VEGF agent, wherein the anti-VEGF agent is a fusion protein with improved efficacy for binding to VEGF and heparin. In embodiments, the present invention provides an anti-VEGF agent, wherein the anti-VEGF agent is a fusion protein with very low endotoxin levels.

In embodiments, the present invention provides an anti-VEGF agent, wherein the anti-VEGF agent is an IgG chimeric protein comprising elements of VEGF receptors. In embodiments, the present invention provides an IgG chimeric protein, wherein the IgG chimeric protein comprises one or more fragments of the seven immunoglobulin (Ig)-like domains in the extracellular portion of VEGF tyrosine kinase receptors. In embodiments, the present invention provides an IgG chimeric protein, wherein the IgG chimeric protein comprises one or more extracellular domain fragments of VEGFR-1 fused with Fc-IgG. In embodiments, the present invention provides an IgG chimeric protein comprising at least one VEGF binding domain VEGFR-1 domain 2 and at least one additional VEGFR-1 domain 1 or 3, and not including domain 4. In embodiments, the present invention provides an IgG chimeric protein, wherein the IgG chimeric protein comprises one or more extracellular domain fragments of VEGFR-2 fused with Fc-IgG. In embodiments, the present invention provides an IgG chimeric protein, wherein the IgG chimeric protein comprises one or more extracellular domain fragments of VEGFR-1 and VEGFR-2 fused with Fc-IgG.

In embodiments, the present invention provides an anti-VEGF agent comprising a VEGF binding portion operatively linked to a Fc-IgG, wherein the VEGF binding portion comprises at least one VEGF binding domain that is an IgG-like domain 2 of VEGFR-1, and wherein the anti-VEGF agent has a VEGF-stimulated mitogenesis-inhibiting ability greater than aflibercept. In embodiments, the present invention provides that the anti-VEGF agent has a vitreous binding ability greater than aflibercept. In embodiments, the present invention provides that the anti-VEGF agent has a vitreous bound VEGF-stimulated endothelial cell proliferation-inhibiting ability greater than aflibercept. In embodiments, the present invention provides that the agent has an increased half-life in vivo compared to aflibercept.

In embodiments, the present invention provides that the VEGF binding portion consists essentially of IgG-like domains 1, 2, and 3 of VEGFR-1 (V₁₋₂₋₃). In embodiments, the anti-VEGF agent comprises amino acid sequence as defined in SEQ ID NO: 1.

In embodiments, the present invention provides that the VEGF binding portion consists essentially of IgG-like domains 2 and 3 of VEGFR-1 (V₂₋₃). In embodiments, the anti-VEGF agent comprises amino acid sequence as defined in SEQ ID NO: 3.

In embodiments, the present invention provides that the VEGF binding portion consists essentially of IgG-like domains 1, 2, 3 and 3 of VEGFR-1 (V₁₋₂₋₃₋₃). In embodiments, the anti-VEGF agent comprises amino acid sequence as defined in SEQ ID NO: 5.

In embodiments, the present invention provides that the VEGF binding portion consists essentially of IgG-like domains 2, 3 and 3 of VEGFR-1 (V₂₋₃₋₃). In embodiments, the anti-VEGF agent comprises amino acid sequence as defined in SEQ ID NO: 7.

In embodiments, the present invention provides pharmaceutical compositions comprising a therapeutically effective amount of an anti-VEGF agent as defined claims and a pharmaceutically acceptable excipient. In embodiments, the present invention provides methods of treating a VEGF-related disorder in a subject in need comprising administering to the subject a therapeutically effective amount of an anti-VEGF agent as defined. The anti-VEGF agent can be directly injected into the affected tissue or organ, such as an eye.

In embodiments, the present invention provides a method for treating ocular disease, wherein an anti-VEGF agent is administered locally to the eye at a dosage corresponding to a molar ratio of 2:1 compared to VEGF. In embodiments, the present invention provides a method for treating ocular disease, wherein an anti-VEGF agent is administered by intravitreous injection. In embodiments, the present invention provides a method for treating ocular disease, wherein an anti-VEGF agent is administered every 4-6 weeks, and in other embodiments, the treatment is continued for a period of at least one year.

According to one embodiment, the present invention provides a method for treating ocular disease comprising administering a therapeutically effective amount of an anti-VEGF agent locally into the eye wherein the treatment is effective to treat occult, minimally classic, and predominantly classic forms of wet macular degeneration, wherein the agent is a fusion protein.

In embodiments the invention can be used to treat a wide variety of VEGF-related disorders including neovascular age related macular degeneration, choroidal neovascularization secondary to myopia, proliferative diabetic retinopathy, diabetic macular edema, retinal vascular obstruction such as retinal vein occlusion, ocular tumors, von Hippel Lindau syndrome, retinopathy of prematurity, polypoid choroidal vasculopathy, colorectal cancer, lung cancer, cervical cancer, endometrial cancer, ovarian cancer, kidney cancer, schwannomas, gliomas, ependimomas, and neoplastic or non-neoplastic disorders that benefit from anti-VEGF therapy.

According to another aspect, the present invention provides a pharmaceutical formulation comprising an anti-VEGF agent in a pharmaceutically acceptable carrier formulation for local administration such as into the eye.

In embodiments, the present invention discloses novel constructs, wherein the constructs potently neutralize the activity of VEGF while, at the same time, have strong heparin-binding characteristics.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic representation of exemplary constructed fusion proteins with various Ig-like extracellular domains of VEGFR-1 (V) fused to Fc-IgG (Fc). The following constructs are shown: V₁₋₂₋₃-Fc; V₂₋₃-Fc; V₁₋₂₋₃₋₃-Fc; V₂₋₃₋₃-Fc; V₁₋₂₋₃₋₄-Fc; V₂₋₃₋₄-Fc; V₁₋₂₋₄-Fc and V₂₋₄-Fc.

FIG. 2 depicts a strategy of plasmid construction and expression.

FIG. 3 depicts the amino acid sequence and nucleic acid sequence of construct V₁₋₂₋₃. SEQ ID NO: 1 and SEQ ID NO: 2, respectively.

FIG. 4 depicts the amino acid sequence and nucleic acid sequence of construct V₂₋₃. SEQ ID NO: 3 and SEQ ID NO: 4, respectively.

FIG. 5 depicts the amino acid sequence and nucleic acid sequence of construct V₁₋₂₋₃₋₃. SEQ ID NO: 5 and SEQ ID NO: 6, respectively.

FIG. 6 depicts the amino acid sequence and nucleic acid sequence of construct V₂₋₃₋₃. SEQ ID NO: 7 and SEQ ID NO: 8, respectively.

FIG. 7 depicts the amino acid sequence and nucleic acid sequence of construct V₁₋₂₋₃₋₃₋₄. SEQ ID NO: 9 and SEQ ID NO: 10, respectively.

FIG. 8 depicts the amino acid sequence and nucleic acid sequence of construct V₂₋₃₋₄. SEQ ID NO: 11 and SEQ ID NO: 12, respectively.

FIG. 9 depicts the amino acid sequence and nucleic acid sequence of construct V₂₋₄. SEQ ID NO: 13 and SEQ ID NO: 14, respectively.

FIG. 10 depicts the expression of VEGFR-1 constructs in 293 cells.

FIG. 11 depicts silver-stained PAGE gels under reducing and non-reducing conditions of 200 ng of each VEGFR-1 Fc fusion protein compared to EYLEA®.

FIGS. 12A-12I depict inhibitory effects of VEGF receptor chimeric proteins on VEGF-stimulated endothelial cell proliferation. Results are provided for V123 (FIG. 12A), V23 (FIG. 12B), V1233 (FIG. 12C), V233 (FIG. 12D), V1234 (FIG. 12E), V234 FIG. 12F), V124 (FIG. 12G), V24 (FIG. 12H), and EYLEA® (FIG. 12I).

FIGS. 13A-13I depict competition of VEGF for Biotinylated VEGF (at 100 ng/ml) binding to VEGFR1 soluble receptor. Results are provided for V123 (FIG. 13A), V1234 (FIG. 13B), V1233 (FIG. 13C), V234 (FIG. 13D), V124 (FIG. 13E), V24 (FIG. 13F), V233 (FIG. 13G), V23 (FIG. 13H), and EYLEA® (FIG. 13I).

FIG. 14 depicts VEGFR-1 soluble receptor binding to Biotinylated VEGF and bovine vitreous.

FIG. 15 depicts bovine vitreous-bound V₁₋₂₋₃₋₃ is biologically active.

FIG. 16 shows effects of control IgG, EYLEA®, or VEGFR-1 Fc fusion proteins on choroidal neovascularization (CNV) area. Each protein was injected intravitreally in the mouse at the dose of 2.5 mg one day before laser treatment. EYLEA® was tested also at 25 mg. Asterisks denote significant differences (Student's t test) compared to the appropriate IgG control groups (**p<0.01, *p<0.05).

FIGS. 17A and 17B show effects of EYLEA®, V_{1,2,3,3} or control IgG on CNV area following a single intravitreal administration (2.5 mg), 1 day, 7 days or 14 days before laser treatment. Asterisks denote significant differences (p<0.05, Student's t test) compared to the IgG control group. FIG. 17B shows representative CD31 immunofluorescence images of groups in FIG. 17A.

### DETAILED DESCRIPTION

All publications, patents, and patent applications mentioned in this specification are incorporated herein by reference to the same extent as if each individual publication, patent, or patent application was specifically and individually indicated to be incorporated by reference.

It is understood that aspects and embodiments of the invention described herein include “consisting” and/or “consisting essentially of” aspects and embodiments. Other objects, advantages and features of the present invention will become apparent from the following specification taken in conjunction with the accompanying figures.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

As used herein, the term “pharmaceutical composition” contemplates compositions comprising one or more therapeutic agents or drugs as described below, and one or more pharmaceutically acceptable excipients, carriers, or vehicles.

As used herein, the term “pharmaceutically acceptable excipients, carriers, or vehicles” comprises any acceptable materials, and/or any one or more additives known in the art. As used herein, the term “excipients,” “carriers,” or “vehicle” refer to materials suitable for drug administration through various conventional administration routes known in the art. Excipients, carriers, and vehicles useful herein include any such materials known in the art, which are nontoxic and do not interact with other components of the composition in a deleterious manner, and generally refers to an excipient, diluent, preservative, solubilizer, emulsifier, adjuvant, and/or vehicle with which an active agent or drug is administered. Such carriers may be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art.

As used herein, the term “therapeutically effective amount” refers to those amounts that, when administered to a particular subject in view of the nature and severity of that subject's condition, will have a desired therapeutic effect, e.g., an amount which will cure, prevent, inhibit, or at least partially arrest or partially prevent a target condition. In some embodiments, the term “therapeutically effective amount” or “effective amount” refers to an amount of a therapeutic agent or drug that when administered alone or in combination with an additional therapeutic agent or drug to a cell, tissue, organ, or subject is effective to prevent or ameliorate ocular diseases and cancers, including, but not limited to, age-related macular degeneration, proliferative diabetic retinopathy, retinal vein occlusion, choroidal neovascularization secondary to myopia; retinopathy of prematurity, diabetic macular edema, polypoidal choroidal vasculopathy, colorectal cancer, lung cancer, breast cancer, pancreatic cancer, and prostate cancer. A therapeutically effective dose further refers to that amount of the therapeutic agent or drug sufficient to result in amelioration of symptoms, e.g., treatment, healing, prevention, or amelioration of the relevant medical condition, or an increase in rate of treatment, healing, prevention, or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

As used herein, the terms “treating,” “treatment,” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent or drug, the subject shows observable and/or measurable reduction in or absence of one or more signs and symptoms of the particular condition. Reduction of the signs or symptoms of a condition may also be felt by the subject. A subject is also considered treated if the subject experiences stable condition. In some embodiments, treatment with a therapeutic agent or drug is effective to result in the subjects being symptom-free 3 months after treatment, preferably 6 months, more preferably one year, even more preferably 2 or more years post treatment. These parameters for assessing successful treatment and improvement in the condition are readily measurable by routine procedures familiar to a physician of appropriate skill in the art.

As used herein, “preventative” treatment is meant to indicate a postponement of development of a condition or a symptom of a condition, suppressing symptoms that may appear, or reducing the risk of developing or recurrence of a condition or symptom. “Curative” treatment includes reducing the severity of or suppressing the worsening of an existing symptom, or condition.

As used herein, the term “therapeutic agent,” “anti-VEGF agent,” “fusion protein,” “chimeric protein,” or “recombinant protein” comprises a first polypeptide operatively linked to a second polypeptide, wherein the “therapeutic agent,” “anti-VEGF agent,” “fusion protein,” “chimeric protein,” or “recombinant protein” inhibits the activity of VEGF. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art. In some embodiments the term “therapeutic agent,” “fusion protein,” “chimeric protein,” or “recombinant protein” refers to any constructs expressed or synthesized, including but not limited to, peptides or proteins operatively linking one or more of the Ig-like domains or domain fragments of VEGFR-1 and/or VEGFR-2 with Fc-IgG.

The term “Ig-like domains” refers to Ig-like domains 1-7 of VEGFR-1 and VEGFR-2. The term “Ig-like domain fragments” comprise a portion of a full-length domain, generally the heparin and/or VEGF binding or variable region thereof. Examples of domain fragments include amino acid sequences comprising a segment of at least 75%, more preferably at least 80%, 90%, 95%, and most preferably 99% of the full-length domain with 100% sequence identity and variations thereof. Variations in the amino acid sequences of fusion proteins are contemplated as being encompassed by the present disclosure, providing that the variations in the amino acid sequence maintain at least 75%, more preferably at least 80%, 90%, 95%, and most preferably 99%. Certain percentages in between are included, such as 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, and 99% sequence identity. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional fusion protein can readily be determined by assaying the specific activity of the fusion protein derivative. Fragments or analogs of fusion proteins can be readily prepared by those of ordinary skill in the art. Preferred amino- and carboxy-termini of fragments or analogs occur near boundaries of functional domains.

As used herein, an “isolated” or “purified” fusion protein means the fusion protein is the predominant species present (i.e., on a molar basis it is more abundant than any other individual species in the composition), and preferably a substantially purified fraction is a composition wherein the fusion protein comprises at least about 50% (on a molar basis) of all macromolecular species present. Generally, a purified composition will comprise more than about 80% of all macromolecular species present in the composition, more preferably more than about 85%, 90%, 95%, and 99%. Most preferably, the fusion protein is purified to essential homogeneity (contaminant species cannot be detected in the composition by conventional detection methods) wherein the composition consists essentially of a single macromolecular species.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

In one aspect the present invention discloses a composition comprising a therapeutic agent, where the therapeutic agent comprises one or more heparin binding domains of VEGFR-1 or VEGFR-2, and one or more VEGF binding domains, thereby inhibiting the binding of VEGF to its cognate receptor.

In embodiments, the present invention provides an anti-VEGF agent comprising a VEGF binding portion operatively linked to a Fc-IgG, wherein the VEGF binding portion comprises at least one VEGF binding domain that is an IgG-like domain 2 of VEGFR-1, and wherein the anti-VEGF agent has a VEGF-stimulated mitogenesis-inhibiting ability greater than aflibercept. In embodiments, the present invention provides that the anti-VEGF agent has a vitreous binding ability greater than aflibercept. In embodiments, the present invention provides that the anti-VEGF agent has a vitreous bound VEGF-stimulated endothelial cell proliferation-inhibiting ability greater than aflibercept. In embodiments, the present invention provides that the agent has an increased half-life in vivo compared to aflibercept.

In embodiments, the present invention provides that the VEGF binding portion consists essentially of IgG-like domains 1, 2, and 3 of VEGFR-1 (V₁₋₂₋₃). In embodiments, the anti-VEGF agent comprises amino acid sequence as defined in SEQ ID NO: 1.

In embodiments, the present invention provides that the VEGF binding portion consists essentially of IgG-like domains 2 and 3 of VEGFR-1 (V₂₋₃). In embodiments, the anti-VEGF agent comprises amino acid sequence as defined in SEQ ID NO: 3.

In embodiments, the present invention provides that the VEGF binding portion consists essentially of IgG-like domains 1, 2, 3 and 3 of VEGFR-1 (V₁₋₂₋₃₋₃). In embodiments, the anti-VEGF agent comprises amino acid sequence as defined in SEQ ID NO: 5.

In embodiments, the present invention provides that the VEGF binding portion consists essentially of IgG-like domains 2, 3 and 3 of VEGFR-1 (V₂₋₃₋₃). In embodiments, the anti-VEGF agent comprises amino acid sequence as defined in SEQ ID NO: 7.

In embodiments, the present invention provides pharmaceutical compositions comprising a therapeutically effective amount of an anti-VEGF agent as defined claims and a pharmaceutically acceptable excipient. In embodiments, the present invention provides methods of treating a VEGF-related disorder in a subject in need comprising administering to the subject a therapeutically effective amount of an anti-VEGF agent as defined. The anti-VEGF agent can be directly injected into the affected tissue or organ, such as an eye.

In embodiments the invention can be used to treat a wide variety of VEGF-related disorders including neovascular age related macular degeneration, choroidal neovascularization secondary to myopia, proliferative diabetic retinopathy, diabetic macular edema, retinal vascular obstruction such as retinal vein occlusion, ocular tumors, von Hippel Lindau syndrome, retinopathy of prematurity, polypoid choroidal vasculopathy, colorectal cancer, lung cancer, cervical cancer, endometrial cancer, ovarian cancer, kidney cancer, schwannomas, gliomas, ependimomas, and neoplastic or non-neoplastic disorders that benefit from anti-VEGF therapy.

In some embodiments, the therapeutic agent is in an administrable dosage form, comprising the therapeutic agent, and an additional excipient, carrier, adjuvant, solvent, or diluent.

In some embodiments, the present invention discloses a pharmaceutical composition suitable for treating and/or preventatively treating a subject, wherein the therapeutic agent is contained in an amount effective to achieve its intended purpose.

In some embodiments, the therapeutic agent or compositions disclosed herein are administered by injection. In certain embodiments, the compositions or the therapeutic agent are injected directly into the diseased organ or tissue. In some embodiments, the therapeutic agent can be topically administered, for example, by patch or direct application to the diseased organ or tissue, or by iontophoresis. The therapeutic agents may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained released composition may be appropriate.

The therapeutic agent may also be delivered using an implant, such as but not limited to an intraocular implant. Such implants may be biodegradable and/or biocompatible implants, or may be non-biodegradable implants. The implants may be permeable or impermeable to the active agent. The specific implants for delivery of the therapeutic agent is dependent on both the affected tissue or organ as well as the nature of the condition being treated. The use of such implants is well known in the art.

The inhibitors described in this invention can be formulated in nanoparticles or other drug formulations in order to provide precise delivery to specific tissues and also provide controlled release therapy.

The inhibitors described in this application can be delivered not only as purified recombinant proteins but also by a gene therapy approach. Recombinant adeno-associated vectors (rAAVs) or other suitable vectors can be used to deliver the VEGF inhibitor by sub-retinal or intravitreal delivery^{43,44}.

In a related aspect, the present invention provides a method for treating a VEGF-related or neovascular disorder in a subject, wherein the method involves administering to the subject: (a) an effective amount of a fusion protein capable of binding heparin and diminishing or preventing the development of unwanted neovasculature. The fusion protein may be combined with other anti-VEGF agents including, but are not limited to: antibodies or antibody fragments specific to VEGF; antibodies specific to VEGF receptors; compounds that inhibit, regulate, and/or modulate tyrosine kinase signal transduction; VEGF polypeptides; oligonucleotides that inhibit VEGF expression at the nucleic acid level, for example antisense RNAs; and various organic compounds and other agents with angiogenesis inhibiting activity.

The invention provides that heparin-binding mediated by D3 (or other Ig-like domain) of VEGFR1²⁸, while a disadvantage for systemic administration, can confer important advantages for intravitreal (or other local) administration. Indeed, the ability to bind HGPSG, key components of the extracellular matrix²⁹, promotes accumulation in the vitreous as well as retinal penetration³⁰. The invention provides a series of VEGFR-1 Fc fusion constructs having differential abilities to interact with HSPGs. This enables election of VEGF inhibitors with different duration/half-life in the eye, which are useful under difference clinical conditions.

The features and other details of the invention will now be more particularly described and pointed out in the following examples describing preferred techniques and experimental results. The examples are provided for the purpose of illustrating the invention and should not be construed as limiting.

### EXAMPLES

In embodiments, the present invention therefore discloses anti-VEGF agents that are novel and improve on existing anti-VEGF agents, including aflibercept, owing to high biological potency combined with strong heparin-binding characteristics. The heparin binding is predictive of a longer half-life and consequently reduced frequency of administration.

The invention provides that heparin-binding mediated by D3 (or other Ig-like domain) of VEGFR1²⁸, while a disadvantage for systemic administration, can confer important advantages for intravitreal (or other local) administration. Indeed, the ability to bind HGPSG, key components of the extracellular matrix²⁹, may promote accumulation in the vitreous as well as retinal penetration³⁰. The invention provides a series of VEGFR-1 Fc fusion constructs having differential abilities to interact with HSPGs.

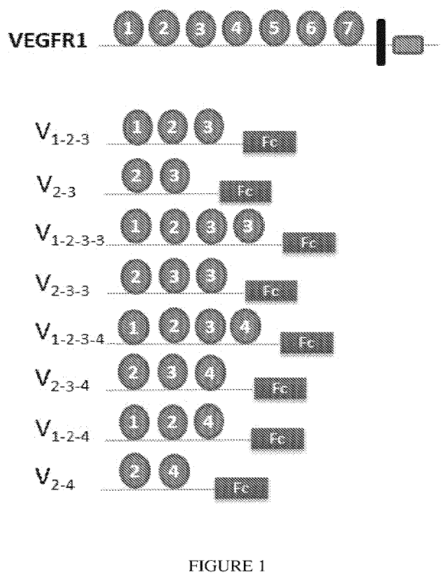

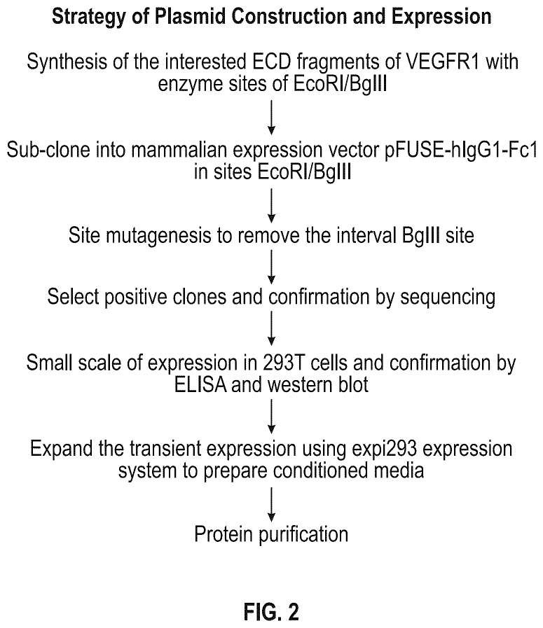

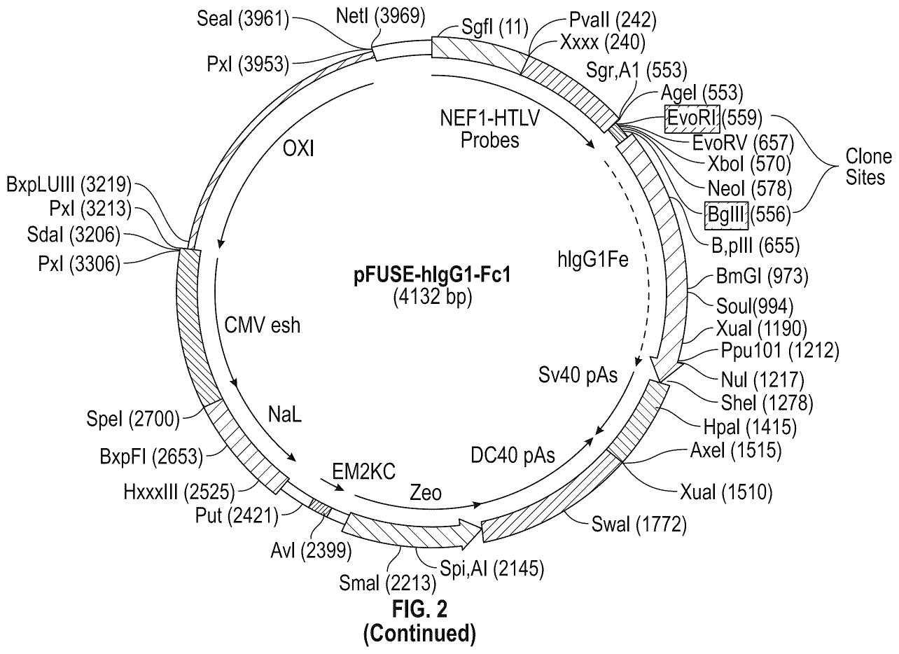

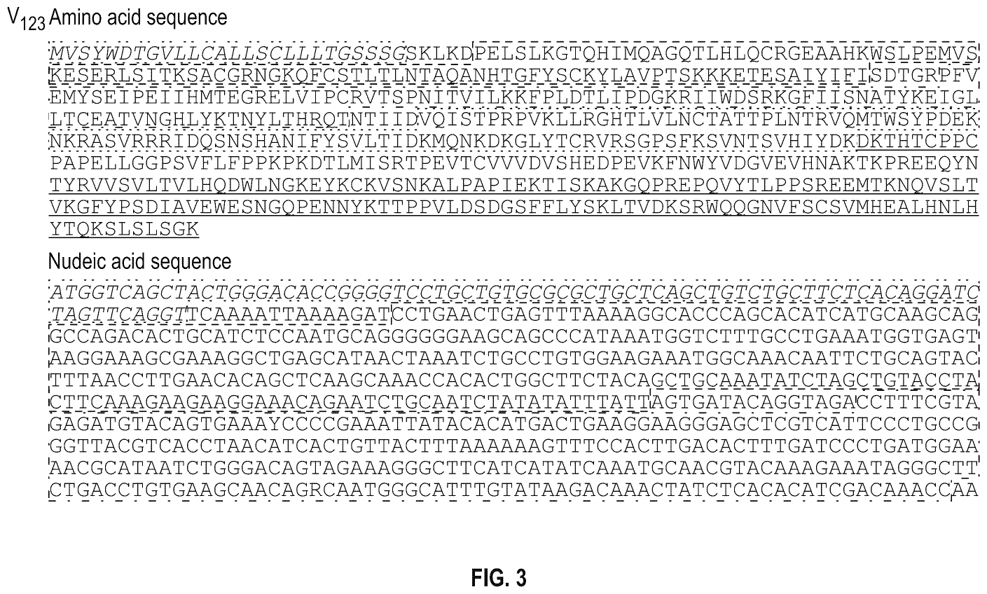

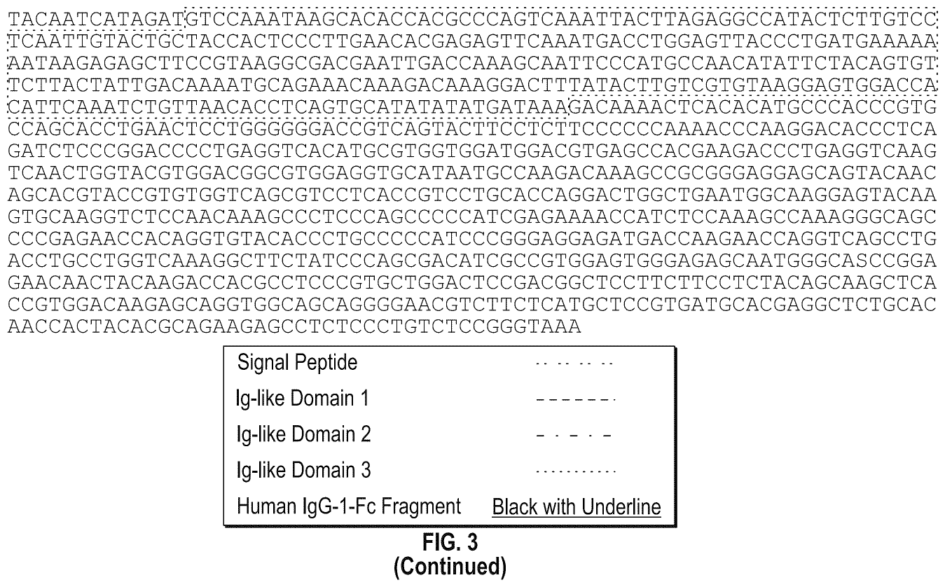

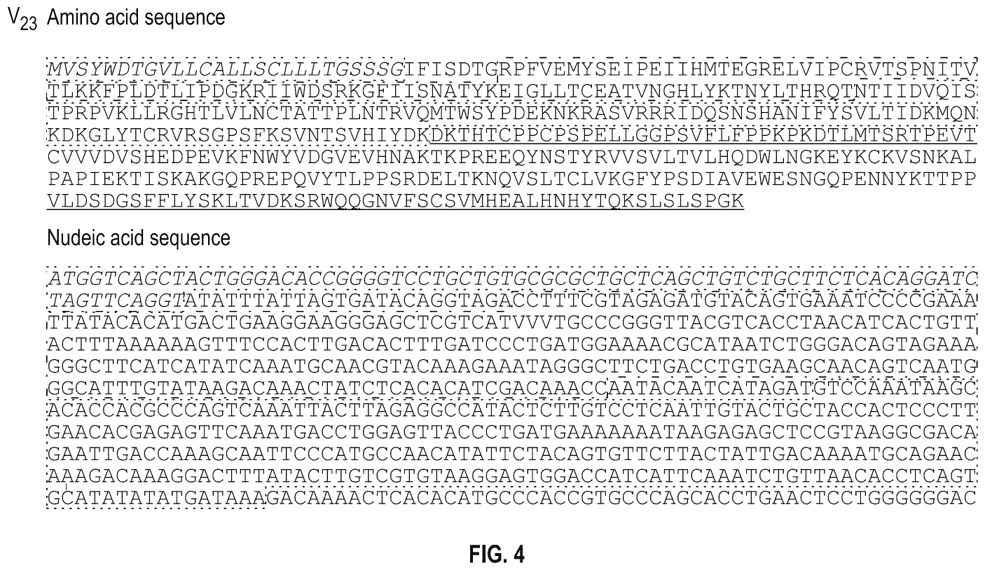

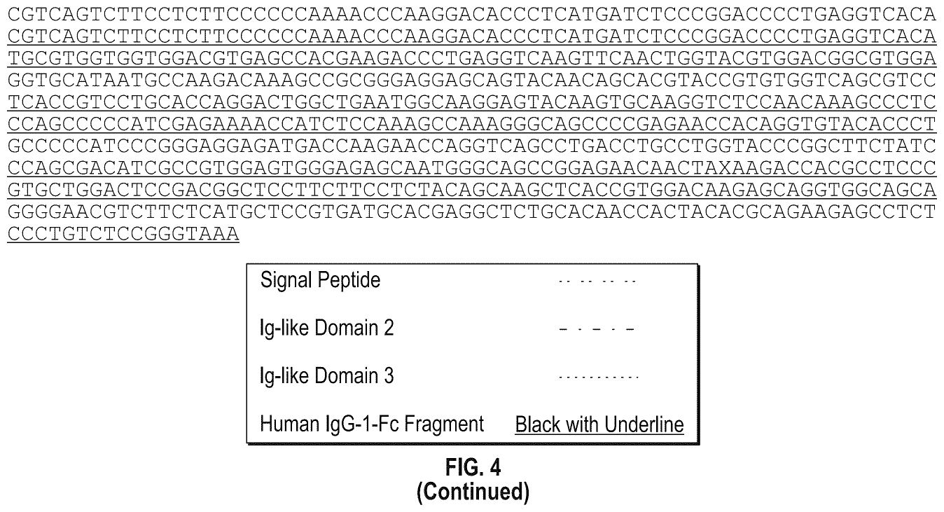

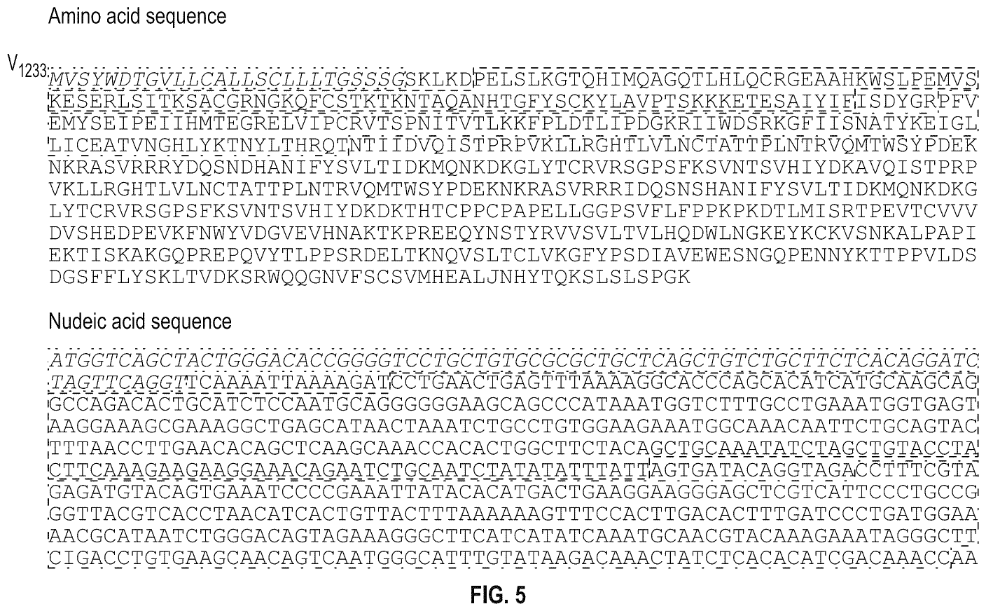

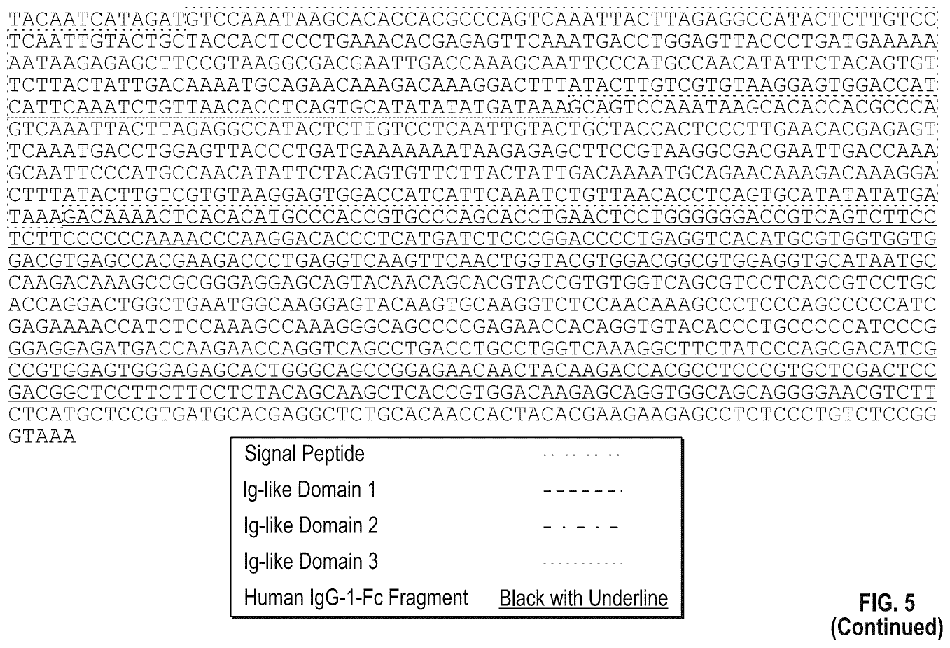

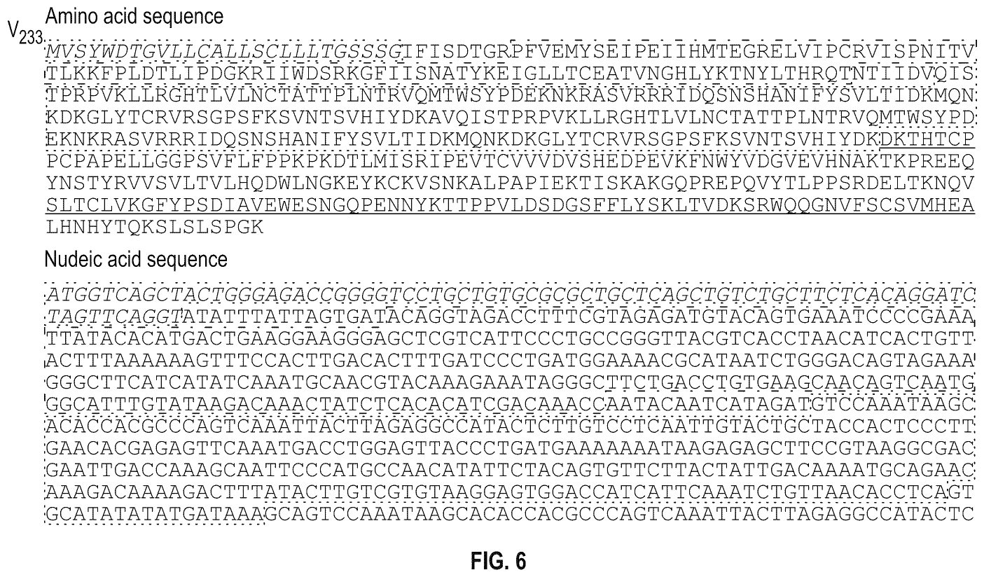

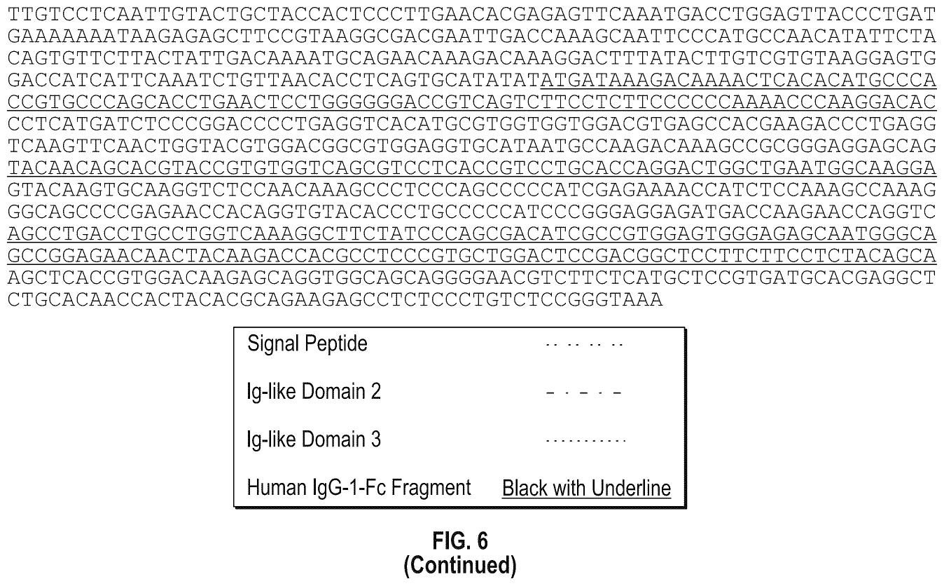

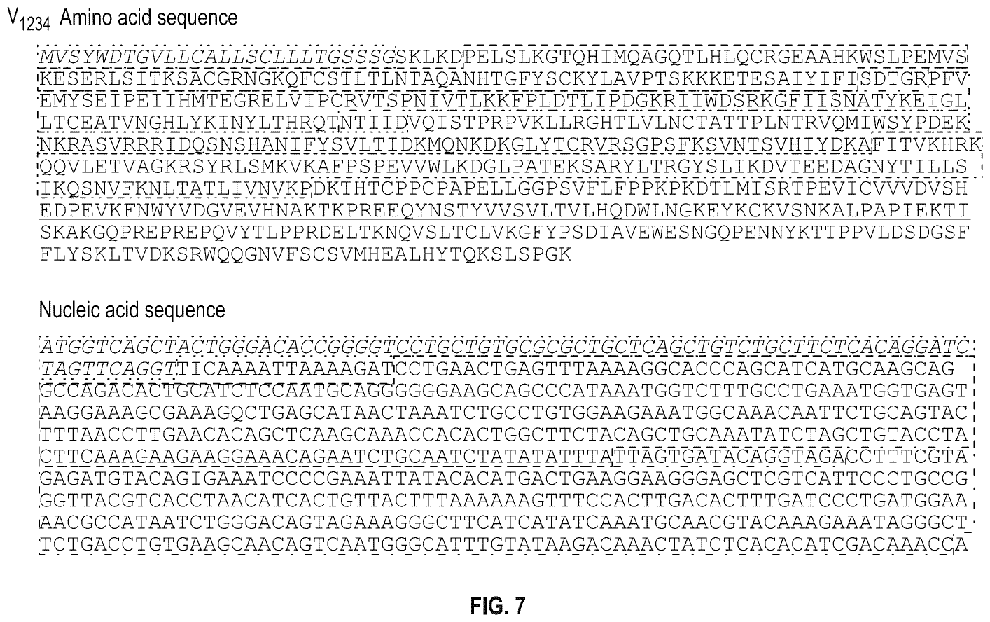

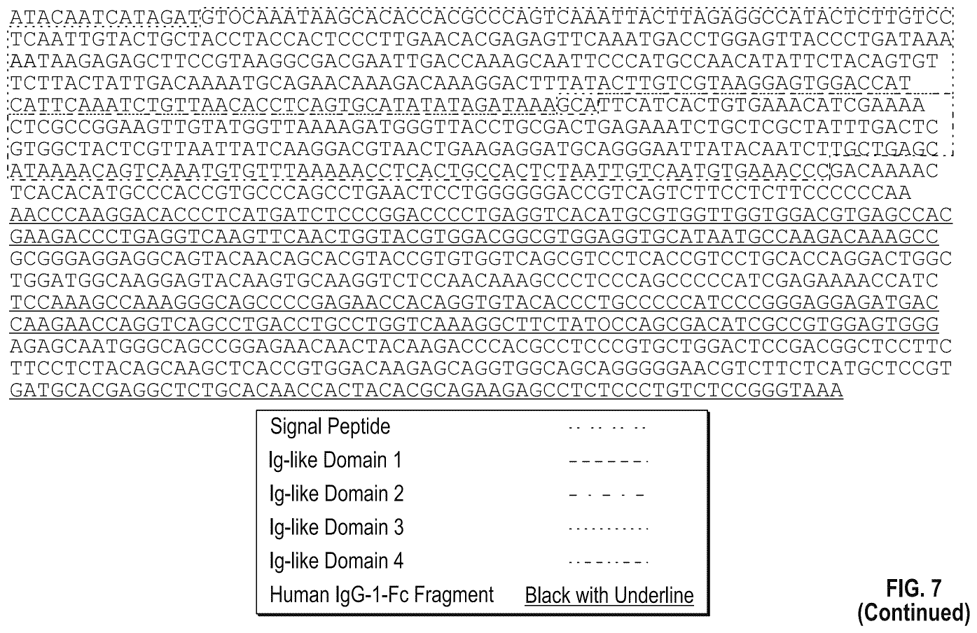

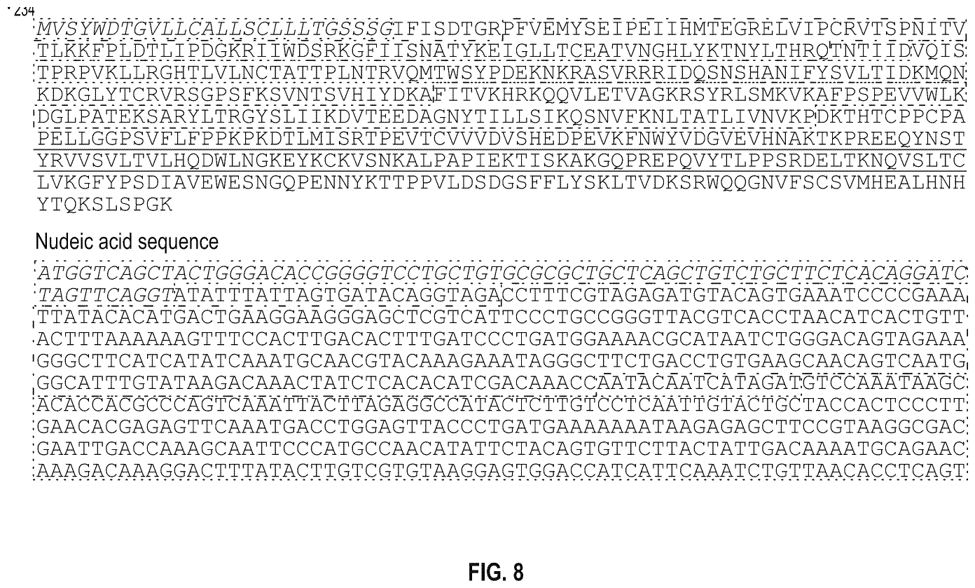

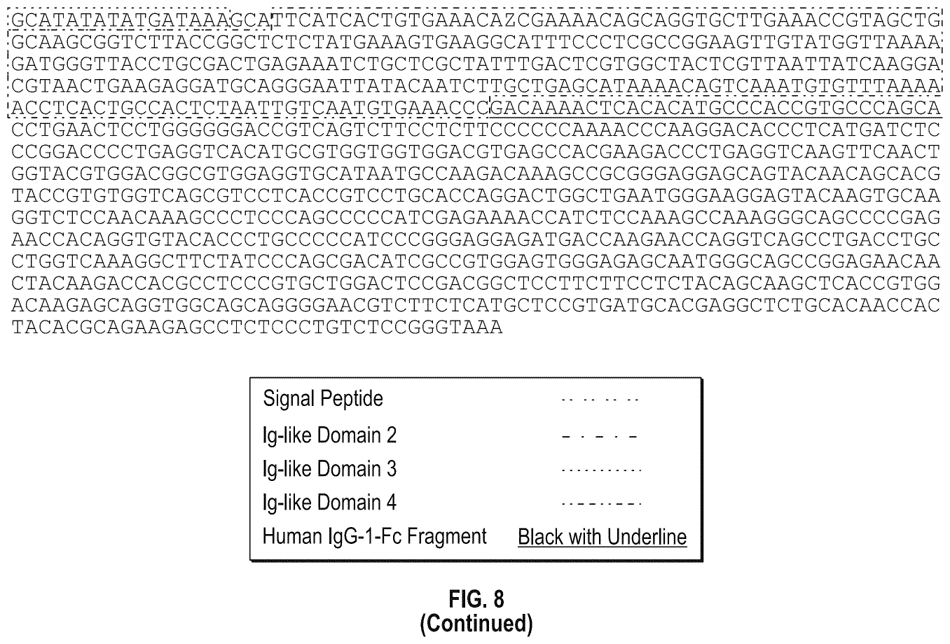

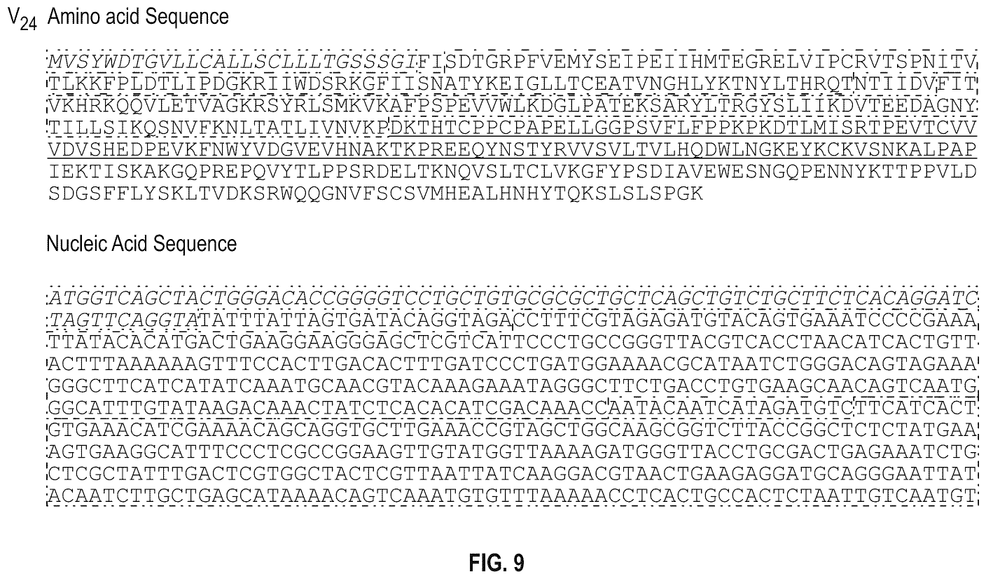

FIG. 1 provides a schematic representation of the constructs employed here. FIG. 2 illustrates the vector employed and the cloning strategy. FIG. 3-9 show the nucleic acid and amino acid sequences of the constructs generated.

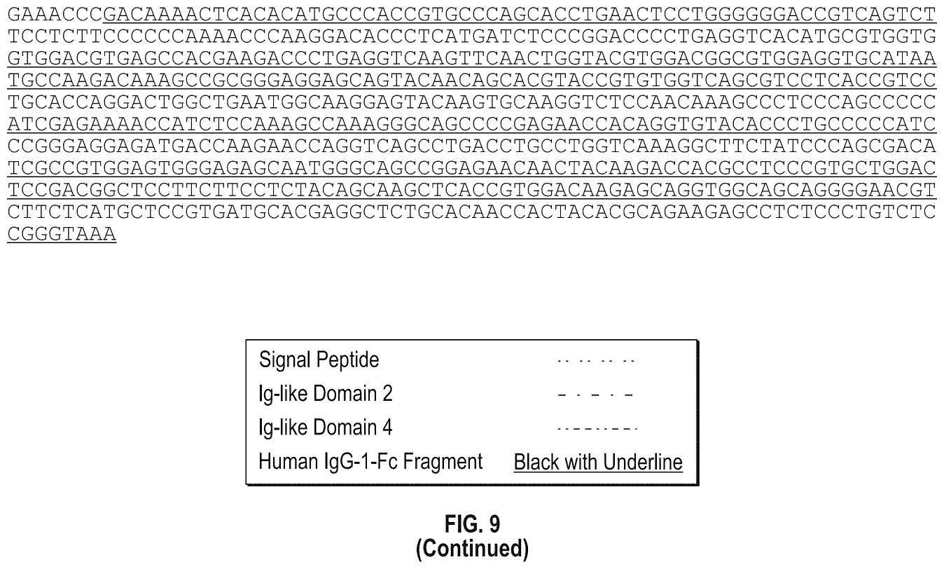

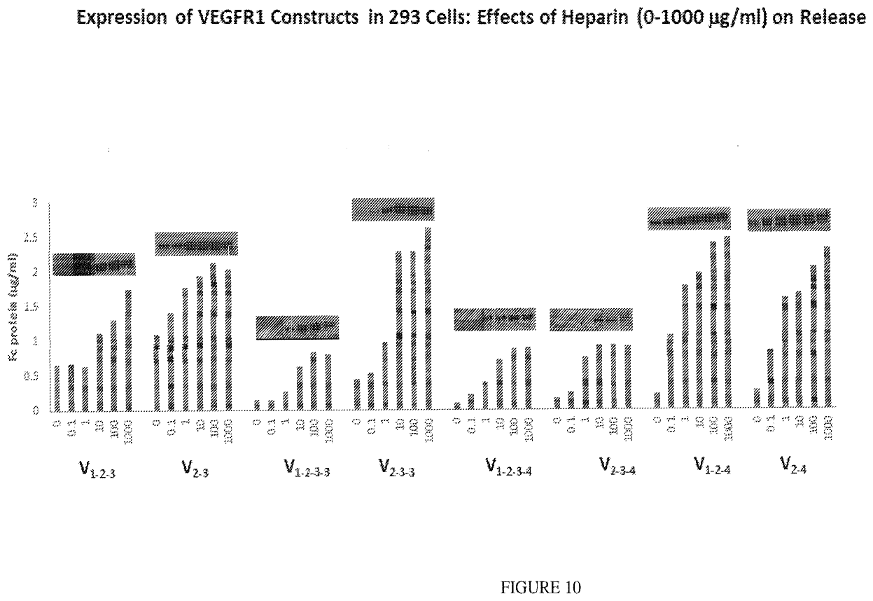

The Examples show that the expression levels of most constructs were low; V₁₋₂₋₃₋₄, V₁₋₂₋₃₋₃, V₂₋₃₋₄ and V₁₋₂₋₄ were almost completely undetectable in the conditioned media. Previous studies had shown that VEGF isoforms with high affinity for heparin (VEGF₁₈₉ or VEGF₂₀₆) are undetectable in the conditioned media of transfected cells, being tightly bound to the cells surface or the extracellular matrix^{31 32}. However, they could be released in a soluble form by the addition of heparin or heparinase, indicating that the binding site consisted of HSPG^{31 32}. This example sought to determine whether the addition of heparin may also affect the levels of recombinant VEGFR-1 fusion proteins. Indeed, adding heparin to the media of transfected cells in 6-well plates resulted in dose-dependent increases in the concentrations of recombinant protein in the media (FIG. 10).

In seeking to purify the recombinant proteins, conventional protein A (PA) affinity chromatography alone yielded a major band of the expected mass, but with numerous minor bands, likely reflecting the interaction of the recombinant proteins with host cell-derived HSPGs and other molecules. A protocol was developed that removed such impurities, as described in Methods. A wash at high pH (for example 9.2) in the presence of 1.2 M NaCl while the protein is bound to PA, resulted in release of numerous contaminants. The next step, anion exchange chromatography with Hi-Trap Q, was very effective at removing the bulk of contaminants and aggregates, while the purified proteins were in the flow-through. The LPS levels in the final purified preparations were <0.1 EU/mg (range 0.02-0.08), a very low level compatible with preclinical studies ³³. As shown in FIG. 11, the purity of recombinant proteins was >95%, as assessed by silver-stained SDS/PAGE gel and was similar to that of the FDA-approved drug EYLEA®.

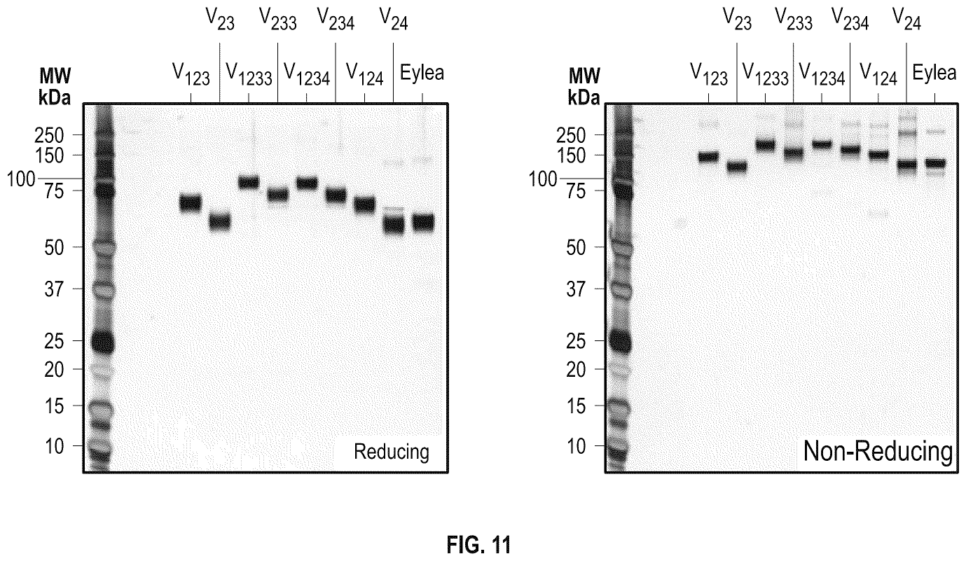

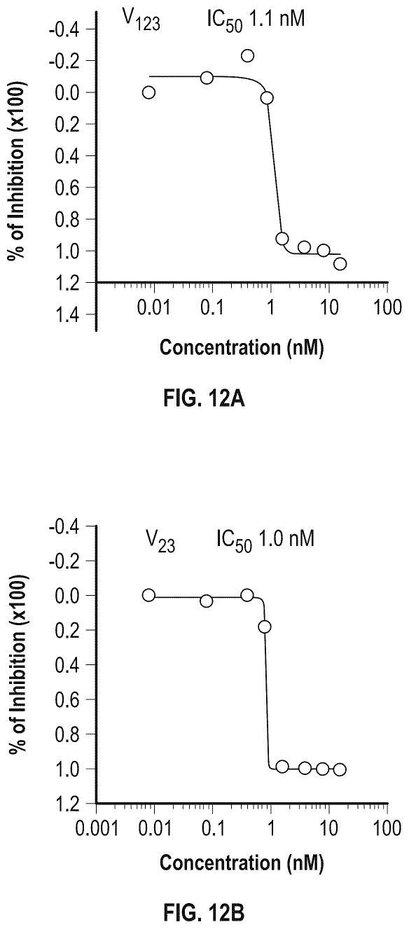

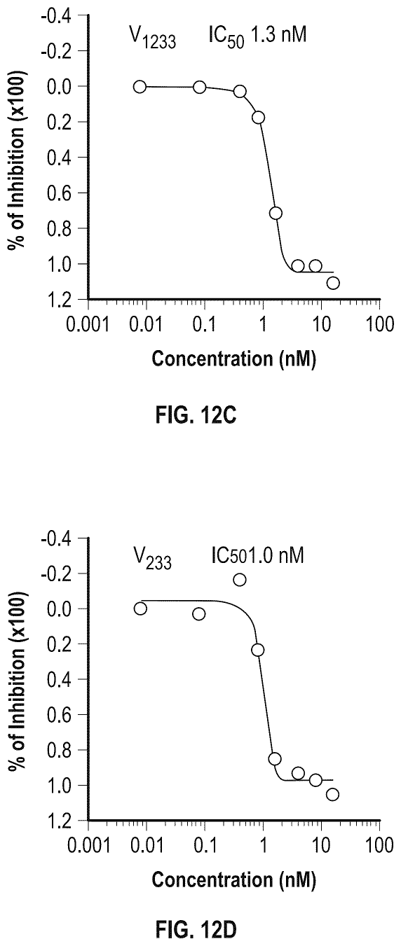

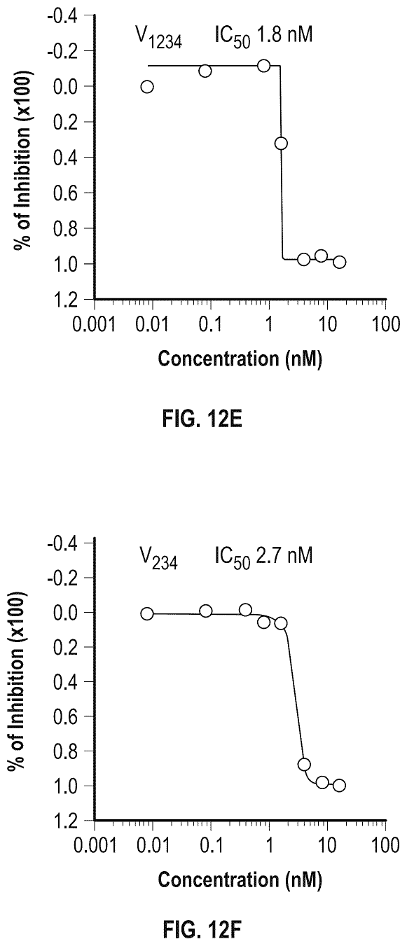

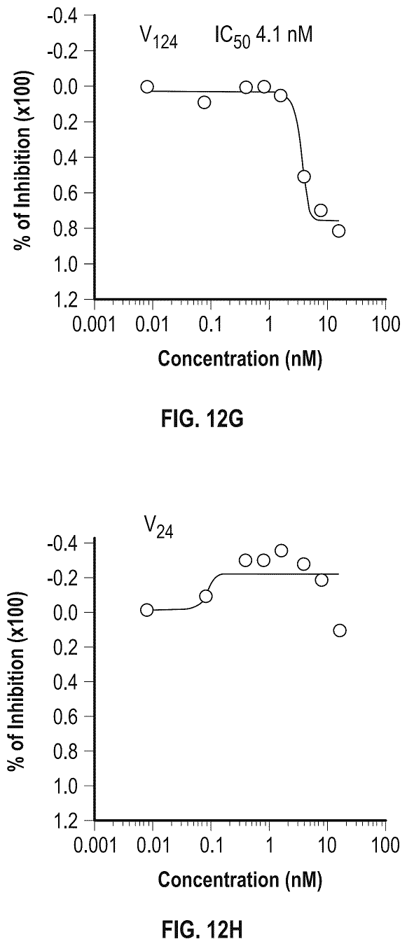

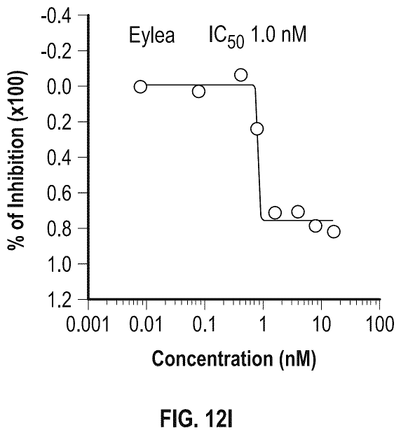

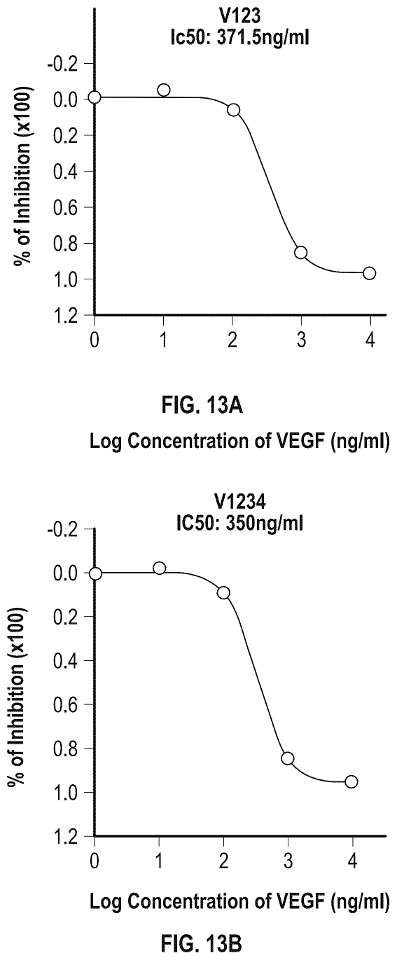

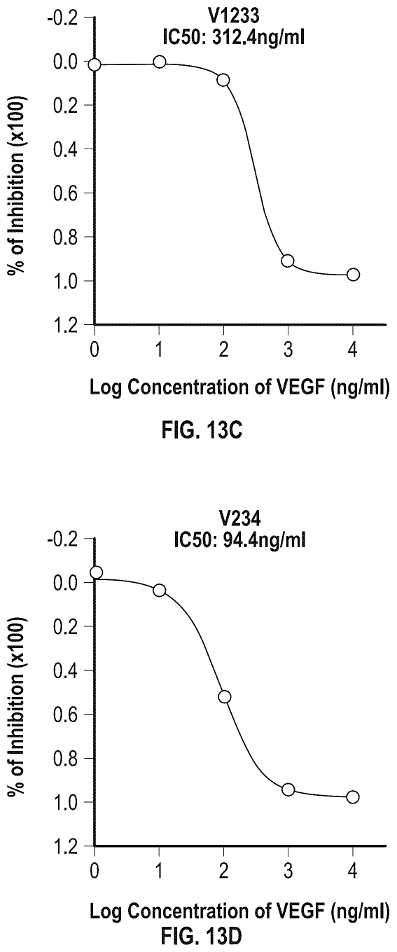

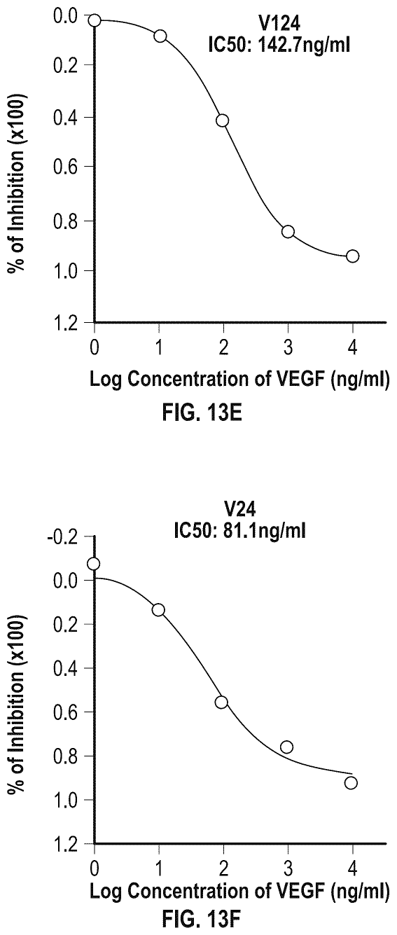

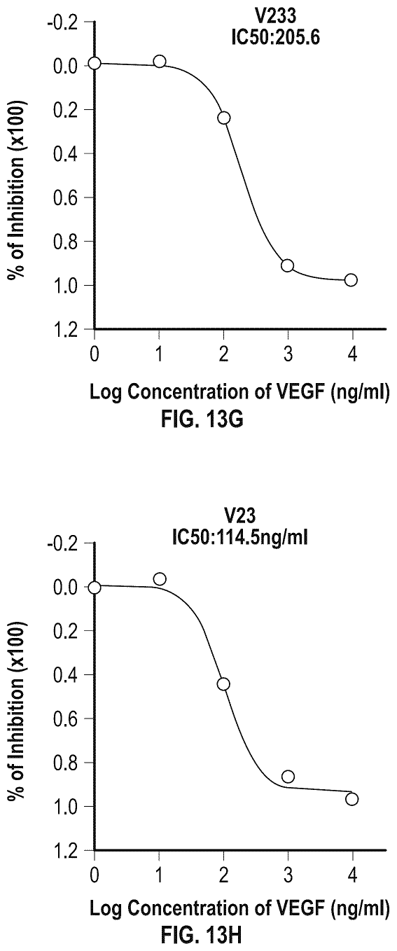

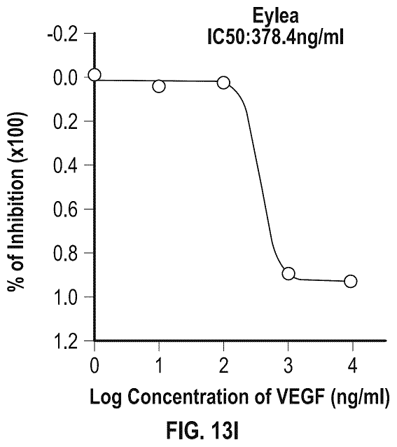

The recombinant proteins were tested for their ability to inhibit mitogenesis stimulated by VEGF (10 ng/ml) in bovine choroidal endothelial cells. The recombinant proteins had inhibitory effects, with IC₅₀ values were in the range of ˜1 nM, except for V1-2-4 and V2-4, which were less potent (FIG. 12). Interestingly, EYLEA®, even at the highest concentration tested, inhibited no more than ˜80% of VEGF-stimulated proliferation (FIG. 12). In contrast, the present VEGFR-1 constructs, (except, V₁₋₂₋₄ and V₂₋₄), completely blocked VEGF-induced proliferation (FIG. 12). Binding assays documented the interaction between the soluble VEGF receptors and the biotinylated VEGF and the ability of VEGF to displace binding (FIG. 13, 14).

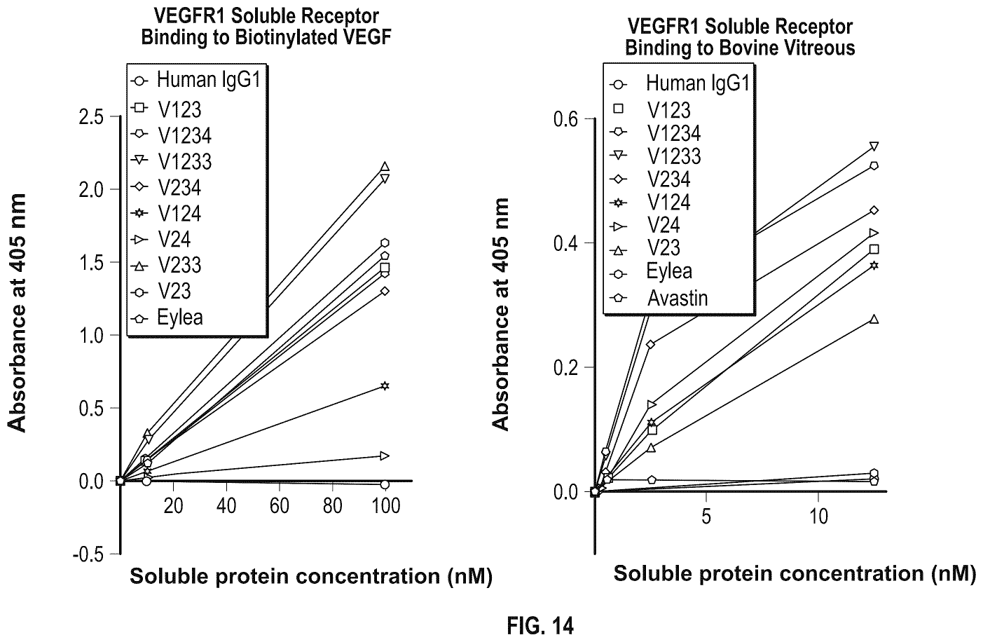

To further define therapeutically relevant interactions, we sought to assess whether the recombinant proteins bind bovine vitreous in vitro. As illustrated in FIG. 14, while EYLEA® or control IgG had little or no binding, the present proteins showed significant binding. The strongest binders were V1-2-3-3, V2-3-3 and V1-2-3-4 followed by V1-2-3. V2-3 had intermediate binding characteristics, between EYLEA® (or control IgG) and V₁₋₂₋₃₋₃. AVASTIN®, a monoclonal antibody⁹ commonly used to treat intraocular neovascularization, also had little or no binding.

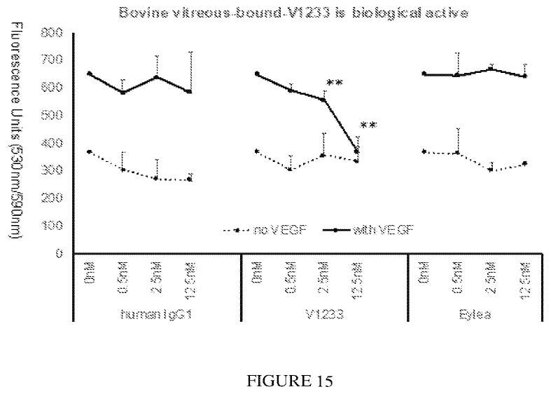

To determine whether vitreous-bound VEGFR-1 Fc fusion may be biologically active, plates were coated with bovine vitreous. Addition of V1-2-3-3, but not EYLEA® or control IgG, inhibited the ability of exogenously added VEGF to stimulate endothelial cell proliferation (FIG. 15).

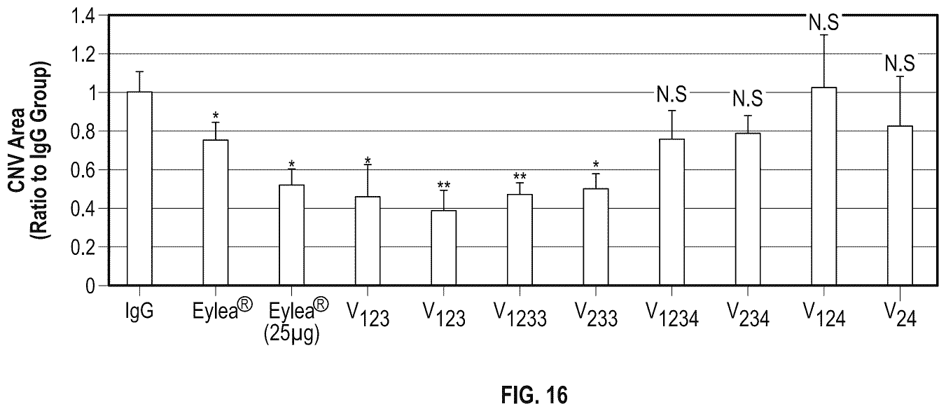

The recombinant proteins were tested in the mouse CNV assay and compared them to control IgG or EYLEA®. Each protein was injected intravitreally at the dose of 2.5 μg one day before laser treatment. EYLEA® was tested also at 25 μg. V₁₋₂₋₃, V₂₋₃, V₁₋₂₋₃₋₃ and V₂₋₃₋₃ at the dose of 2.5 μg demonstrated a degree of inhibition similar or greater to that achieved with 25 μg EYLEA®. However, none of the constructs containing D4 demonstrated significant inhibition under the circumstances tested (FIG. 16).

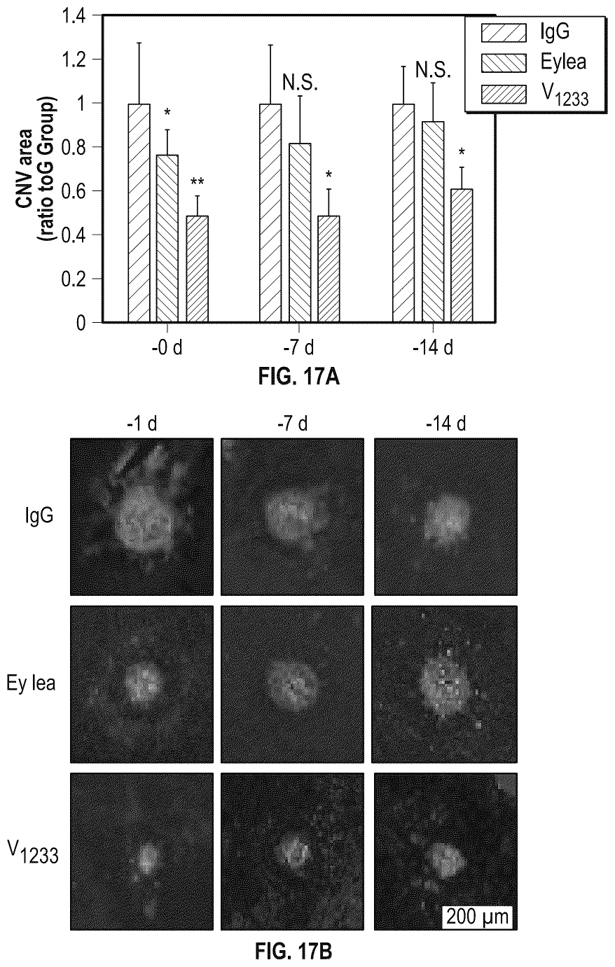

To determine whether heparin binding may translate in durable therapeutic effects following a single administration, V₁₋₂₋₃₋₃, EYLEA® or control IgG, were injected intravitreally (2.5 μg) 1 day, 7 days or 14 days before the laser-induced injury. As shown in FIG. 17, EYLEA® resulted in a significant inhibition only when administered 1 day before the injury. However, V₁₋₂₋₃₋₃ resulted in a significant inhibition also when administered 7 days or 14 days prior to the injury.

Disclosed are several novel VEGFR-1-Fc constructs evaluated in a variety of in vitro and in vivo assays. To purify the recombinant proteins, a multi-step protocol was used. This was dictated to a large degree by the relatively low expression levels in transiently expressing 293 cells, requiring the addition of heparin to the media to improve release. However, the need to use heparin may be in part or entirely obviated by different host cells (e.g., having a different composition of HSPG or mutants thereof) or by higher expression levels such as in amplified, stable cell lines.

All constructs, except V2-4, potently neutralized the activity of VEGF and, at the same time, had strong heparin-binding characteristics, which may predict a long half-life following intravitreal administration. The example documents that these proteins bind to bovine vitreous. The strongest binders were V1-2-3-3, V2-3-3, V1-2-3-4, followed by V1-2-3. V2-3 had significant but lower vitreous binding. Control IgG, EYLEA®, or AVASTIN® had instead minimal binding. One of the strongest vitreous binders, V1-2-3-3, was selected to test the hypothesis that a vitreal matrix-bound VEGFR1 Fc constructs may be biologically active. As shown in FIG. 15, in plates coated with bovine vitreous, addition of V1-2-3-3, but not EYLEA® or control IgG, inhibited the ability of exogenously added VEGF to stimulate endothelial cell proliferation.

Next the recombinant proteins were tested in the mouse CNV model for their ability to inhibit laser-induced neovascularization. EYLEA® was used as a positive control and human IgG1 as a negative control. Relatively low dose was chosen for in vivo testing, being best suited to reveal potency differences among the various constructs. Also, it has been reported that intravitreal administration of relatively high doses of antibodies of the IgG1 isotype may have off-target inhibitory effects, mediated by FcgRI (CD64) and c-Cbl, when injected intravitreally 34. The dose employed should avoid such off-target effects and detect truly specific effects.

As shown in FIG. 16, EYLEA® resulted in an approximately 30% inhibition at the dose of 2.5 μg and ˜50% inhibition at 25 μg. These findings are largely consistent with the published literature. Saishin et al. reported that the intravitreal injection of ˜5 μg aflibercept resulted in ˜30% inhibition of CNV area in the mouse 35. Indeed, the dose of 40 μg is commonly used to achieve a maximal effect in the mouse CNV model 36. An unexpected finding of our study was the greater potency of some of our constructs: V1-2-3, V2-3, V1-2-3-3 and V2-3-3. Administering 2.5 μg of these constructs one day before the injury matched or even exceeded the level of inhibition achieved with 25 μg of EYLEA®. The finding that V1-2-3-3, but not EYLEA®, has significant effect on preventing CNV when administered 7 days or 14 days before the injury (FIG. 17), documents the durability of the effect and the therapeutic value.

An unexpected finding is that none of the constructs containing D4 (V₁₋₂₋₃₋₄, V₂₋₃₋₄, V₁₋₂₋₄, V₂₋₄) resulted in marked inhibition in vivo (at least at the dose tested), in spite of the fact that these molecules (with the exception of V₂₋₄) demonstrated an ability to block VEGF-stimulated mitogenesis in vitro. However, all of these constructs demonstrated a propensity to form multimers or aggregates, as assessed by SDS/PAGE gel under non-reducing conditions (FIG. 11) or size exclusion chromatography (not shown). Although earlier work ³⁷ identified D4 (together with D7) as a requirement for VEGFR-1 dimerization, such effect has been known to be ligand-dependent. Crystal structure studies revealed a loop in D4 responsible for such homotypic interactions ²³. It is conceivable that high concentrations and/or the forced dimerization imposed by the Fc construct may result in ligand-independent interactions, resulting in aggregation. In any event, aggregates are not desirable pharmaceuticals given the possibility of inflammation and immunogenicity ^{38, 39}. Therefore, an aspect of the present invention is the identification of constructs having VEGFR-1 D2/D3, but not D4, in embodiments.

It is noteworthy that well-characterized Fc mutations, well known to the skilled in the art, that reduce effector functions could be useful additions to the invention in order to minimize antibody-dependent cytotoxicity (ADCC) well as interactions with C1q and the initiation of the complement cascade⁴⁰.

In conclusion, aflibercept was designed to eliminate the heparin-binding heparin domain in order to improve systemic half-life for oncological indications. The constructs described in the present study are instead designed to promote binding and retention in the vitreous to ensure more sustained and therapeutically relevant interactions.

### Methods

For construction of VEGFR-1_{ECD}-Fc expression plasmids, the nucleic acid fragments encoded the signal peptide and a variety of extracellular Ig-like domains one to four of VEGRF-1 (Gene ID: 2321) were synthesized by GenScript USA Inc. The variety of the extracellular Ig-like domain constructs is as follows: V₁₂₃ contains D1, 2 and 3; V23, D1 and D2; V₁₂₃₄, D1, 2, 3 and 4; V₁₂₃₃, D1, 2, 3 and 3; V₂₃₄, D 2, 3 and 4; V₁₂₄, D1, 2 and 4; V24, D2 and 4; F7 is ECD2, 2 and 3 and F8 is ECD2 and 3. The synthesized fragments were inserted into pFUSE-hIgG1-Fc vector (Invitrogen, #pfuse-hgifc1) at EcoRI and BgIII sites, generating the plasmids containing the various Flt1 ECDs. Then, using PrineSTAR Mutagenesis Basal Kit (Takara, R046A), the interval amino acid R and S (BgIII site) between the ECDs and the Fc fragment were removed, generating the plasmids (F1-F8) expressing the fusion proteins of Flt1 ECDs with a 227-amino acid human IgG1-Fc.

### Transfection and Conditioned Media Preparation

The Expi293 expression system (Life technologies, A14524) was used to generate the conditioned media for purification, according to the manufacturer's instructions. In brief, Expi293F™ Cells (ThermoFisher) were suspension-cultured in Expi293™ expression medium at 37° C. in a humidified atmosphere with 8% CO2. When the cell density reached to 2.5 million/ml, plasmids DNA and EXPIFECTAMINE™ 293 reagent was mixed, incubated 5 min and added to the cells. The final concentration of the DNA and transfected reagent was 1 μg and 2.7 μl per milliliter respectively. Five hours after transfection, 100 μg/ml Heparin (Sigma, H3149) and protease inhibitor cocktail, 1:400 (Sigma, P1860), were added to the cells. 16 hours after transfection, enhancer reagents 1 and 2 were added. Ninety-six hours after transfection, conditioned media were harvested. Aliquots were tested for Fc fusion proteins concentrations using a human Fc ELISA Kit (Syd Labs, EK000095-HUFC-2) according to the manufacturer's instructions. Protease inhibitors were added (1:500) to the bulk, which was stored at −80° C. until further use.

### Purification of Recombinant Proteins

Pyrogen-free reagents were employed. Prior to use, columns and equipment (Akta Explorer System) were sanitized by exposure to 0.5 N NaOH for approximately 45 minutes. Conditioned media from transfected cells were adjusted to PBS, 0.01% polysorbate (PS) 20. PS20 was added to buffers at all steps. After centrifugation at 20,000×g for 30 minutes, supernatants were subjected to protein A (PA) affinity chromatography using a Hi-Trap MabSelect SuRe (5 ml, GE Healthcare). After loading, the column was washed with 20 mM diethanolamine, pH 9.2, 1.2 M NaCl, prior to elution with 0.1 M citric acid, pH 3.0, which was immediately neutralized. The PA elution pool was then diluted in 20 mM diethanolamine, pH 9.2, and applied to Hi-Trap Q (5 ml, GE Healthcare) anion-exchange column. The bound material was eluted with a gradient of NaCl. The flow-through, which contained the purified recombinant protein, was immediately adjusted to 20 mM Tris, pH 6.8, and then concentrated through binding to HEPARIN-SEPHAROSE™ (Hi-Trap™-HS). After a wash with 0.2-0.45 M NaCl (depending on the construct), the recombinant VEGFR1 fusion protein was eluted with 1 M NaCl. The final polishing step consisted of size-exclusion chromatography (SEC), using, for example, SUPERDEX® 200 Increase, 10/300 GL or Hi-Load 16/600 SUPERDEX® 200 pg, GE Healthcare. Finally, the proteins were buffer-exchanged by dialysis into 10 mM Tris, pH 6.8, 10 mM histidine, 1% trehalose, 40 mM NaCl, 0.01% PS20. To determine endotoxin levels, ToxinSensor Chromogenic LAL Endotoxin Assay Kit (GenScript, L00350) was used according to the manufacturer's protocol.

### Cell Proliferation Assays

Bovine endothelial cell proliferation assays were performed essentially as previously described⁴¹. Log phase growing bovine choroidal endothelial cells (BCEC) (passage <10) were trypsinized, re-suspended and seeded in 96-well plates (no coating) in low glucose DMEM supplemented with 10% bovine calf serum, 2 mM glutamine, and antibiotics, at a density of 1000 cells per well in 200 μl volume. rhVEGF₁₆₅ (Peprotech) was added at the concentration of 10 ng/ml. Aflibercept (EYLEAR) was purchased from a pharmacy. The inhibitors were added to cell at various concentrations, as indicated in the figures, before adding the ligands. After 5 or 6 days, cells were incubated with alamarBlue™ for 4 hr. Fluorescence was measured at 530 nm excitation wavelength and 590 nm emission wavelength.

### Solid-Phase VEGFR-1 Variant Binding Assays

Costar 96-well EIA/RIA stripwells (#2592, Corning Incorporated, Kennebunk, ME) were coated overnight at 4° C. with purified VEGF receptor proteins (250 ng/well) in coating buffer (BIOLEGEND®, San Diego, CA, #421701). Nonspecific binding sites were blocked by incubating the strips with 2% BSA (Sigma, A6003) in PBS for 1 hour at room temperature (RT) after a single wash with ELISA wash buffer (R&D systems 895003). Strips were then washed 3 times, followed by adding biotinylated human VEGF₁₆₅ (G&P Biosciences, Santa Clara, CA) in assay diluent (BIOLEGEND®, #421203) alone or in combination with various concentrations of non-biotinylated human VEGF165 (R&D systems) at 37° C. for 2 hours. After three washes, bound biotinylated human VEGF₁₆₅ to VEGFR1 was detected by incubation with HRP Streptavidin (1:1000, BIOLEGEND®, #405210) for 1 hour at RT. Strips were washed 5 times before incubation with TMB high sensitivity substrate solution (BIOLEGEND®, #421501) for 30 min, and absorbance at 450 nm was measured after adding equal amount of stop solution (BIOLEGEND®, #77316). All experiments were carried out in duplicate wells and repeated for at least two times.

### In Vitro Binding to Bovine Vitreous

Bovine vitreous (In Vision BioResources, Seattle, WA) was thawed at 4° C. Material was first diluted 1:1 with PBS, filtered through 0.22 μm filter, aliquoted and stored at −80° C. Total protein concentration of bovine vitreous material was measured by Pierce BCA protein assay. Costar 96-well EIA/RIA stripwells were coated with bovine vitreous (1 μg/well) for 4 hr at RT, followed by one wash with ELISA wash buffer. Nonspecific binding sites were blocked by adding 2% BSA in PBS for 1 hr at RT, followed by washing three times with 0.01% PBS-T. To each well, 50 μl VEGFR1 or control proteins were added overnight at 4° C. Next day, plates were washed three times with 0.01% PBS-T, followed by incubating with 100 μl AP-conjugated goat anti-human Fc (1:2000, Invitrogen, #A18832)) for 1 hr at RT. Plates were further washed five times with 0.01% PBS-T before adding 50 μl 1 step PNPP substrate (Thermo Scientific, Rockford, IL, #37621) for 15-30 min at RT. OD was measured at 405 nm.

Effects of vitreous bound VEGFR1 on VEGF-stimulated endothelial cell proliferation in Costar 96-well EIA/RIA stripwells were first UV-sterilized for 1 hr, followed by coating with 1 μg/well bovine vitreous, diluted in PBS for 4 hr at RT. Plates were washed with PBS once, blocked with 2% BSA at 4° C., and washed two times with PBS in biosafety hood. Equal amounts of soluble receptors or control IgG were added to plates, diluted in PBS O/N at 4° C. (50 μl/well). Plates were then washed once with PBS, followed by one wash with assay media containing 10% BCS. 100 μl media was added to each well, followed by addition of VEGF at 5 ng/ml or PBS only as no VEGF control. Plates were incubated with VEGF or PBS for 1 hr, followed by adding 100 μl BCEC cell suspension (final 2500 cells/well). 48 hrs later, proliferation was measured by adding ALAMARBLUE™.

### Laser-Induced Choroidal Neovascularization (CNV)

Male C57BL/6J mice (6-8 week) were anesthetized with ketamine/Xylazine cocktail before laser treatment. CNV lesions were induced by laser photocoagulation using a diode laser (IRIDEX, Oculight GL) and a slit lamp (Zeiss) with a spot size of 50 um, power of 180 mW and exposure duration of 100 ms. ^{36, 42}. Four laser burns were typically induced at 3, 6, 9 and 12 O'clock position around the optic disc in each eye. Different constructs or IgG isotype control were injected intravitreally, at the dose of 2.5 μg per eye, in a 1 μl volume. EYLEA® was used as a positive control at 2.5 or 25 μg. One day after injection, laser treatment was conducted and eyes were enucleated and fixed in 4% paraformaldehyde (PFA) for 15 min, 7 days after laser treatment. In a separate set of studies, selected constructs were injected once 1 day, 7 days or 14 days prior to laser treatment. Choroid-sclera complexes and retinas were separated and anti-CD31 immunofluorescence (IF) was performed to evidence the vasculature by whole mount staining of both retina and choroidal tissues. For CD31 IF, rat anti-mouse antibody BD 550274 was diluted 1:100 and incubated overnight at 4° C. After 4-hour incubation with a secondary anti-rat antibody (Life Technologies A11006), whole mounts were imaged at 488 nm. Quantification of neovascularization in lesion area and vascular density in retina was carried out by Image J. P values were assessed by Student's t test (significant change, p<0.05).

### REFERENCES

[1] Folkman J, Klagsbrun M: Angiogenic factors. Science 1987, 235:442-7.
[2] Klagsbrun M, D'Amore P A: Regulators of angiogenesis. Annu Rev Physiol 1991, 53:217-39.
[3] Ferrara N, Gerber H P, LeCouter J: The biology of VEGF and its receptors. Nature Med 2003, 9:669-76.
[4] Ferrara N, Adamis A P: Ten years of anti-vascular endothelial growth factor therapy. Nat Rev Drug Discov 2016, 15:385-403.
[5] Olsson A K, Dimberg A, Kreuger J, Claesson-Welsh L: VEGF receptor signalling—in control of vascular function. Nat Rev Mol Cell Biol 2006, 7:359-71.
[6] Alitalo K, Tammela T, Petrova T V: Lymphangiogenesis in development and human disease. Nature 2005, 438:946-53.
[7] Ferrara N: VEGF and the quest for tumour angiogenesis factors. Nat Rev Cancer 2002, 2:795-803.
[8] Miller J W, Le Couter J, Strauss E C, Ferrara N: Vascular endothelial growth factor a in intraocular vascular disease. Ophthalmology 2013, 120:106-14.
[9] Presta L G, Chen H, O'Connor S J, Chisholm V, Meng Y G, Krummen L, Winkler M, Ferrara N: Humanization of an Anti-Vascular Endothelial Growth Factor Monoclonal Antibody for the Therapy of Solid Tumors and Other Disorders. Cancer Res 1997, 57:4593-9.
[10] Chen Y, Wiesmann C, Fuh G, Li B, Christinger H W, Mckay P, de Vos A M, Lowman H B: Selection and analysis of an optimized anti-VEGF antibody: crystal structure of an affinity-matured Fab in complex with antigen. Journal of Molecular Biology 1999, 293:865-81.
[11] Chamow S M, Ashkenazi A: Immunoadhesins: principles and applications. Trends In Biotechnology 1996, 14:52-60.
[12] Chamow S M, Ryll T, Lowman H B, Farson D: Therapeutic Fc-Fusion Proteins. Wiley Blackwell, 2014.
[13] Holash J, Davis S, Papadopoulos N, Croll S D, Ho L, Russell M, Boland P, Leidich R, Hylton D, Burova E, Ioffe E, Huang T, Radziejewski C, Bailey K, Fandl J P, Daly T, Wiegand S J, Yancopoulos G D, Rudge J S: VEGF-Trap: a VEGF blocker with potent antitumor effects. Proc Natl Acad Sci USA 2002, 99:11393-8.
[14] Nguyen T T, Guymer R: Conbercept (KH-902) for the treatment of neovascular age-related macular degeneration. Expert Rev Clin Pharmacol 2015, 8:541-8.
[15] Comparison of Age-related Macular Degeneration Treatments Trials Research G, Maguire M G, Martin D F, Ying G S, Jaffe G J, Daniel E, Grunwald J E, Toth C A, Ferris F L, 3rd, Fine S L: Five-Year Outcomes with Anti-Vascular Endothelial Growth Factor Treatment of Neovascular Age-Related Macular Degeneration: The Comparison of Age-Related Macular Degeneration Treatments Trials. Ophthalmology 2016, 123:1751-61.
[16] Holz F G, Tadayoni R, Beatty S, Berger A, Cereda M G, Cortez R, Hoyng C B, Hykin P, Staurenghi G, Heldner S, Bogumil T, Heah T, Sivaprasad S: Multi-country real-life experience of anti-vascular endothelial growth factor therapy for wet age-related macular degeneration. Br J Ophthalmol 2015, 99:220-6.
[17] Regula J T, Lundh von Leithner P, Foxton R, Barathi V A, Cheung C M, Bo Tun S B, Wey Y S, Iwata D, Dostalek M, Moelleken J, Stubenrauch K G, Nogoceke E, Widmer G, Strassburger P, Koss M J, Klein C, Shima D T, Hartmann G: Targeting key angiogenic pathways with a bispecific CrossMAb optimized for neovascular eye diseases. EMBO Mol Med 2016, 8:1265-88.
[18] Rodrigues G A, Mason M, Christie L A, Hansen C, Hernandez L M, Burke J, Luhrs K A, Hohman T C: Functional Characterization of Abicipar-Pegol, an Anti-VEGF DARPin Therapeutic That Potently Inhibits Angiogenesis and Vascular Permeability. Invest Ophthalmol Vis Sci 2018, 59:5836-46.
[19] Vorum H, Olesen T K, Zinck J, Hedegaard M: Real world evidence of use of anti-VEGF therapy in Denmark. Curr Med Res Opin 2016:1-32.
[20] Davis-Smyth T, Chen H, Park J, Presta L G, Ferrara N: The second immunoglobulin-like domain of the VEGF tyrosine kinase receptor Flt-1 determines ligand binding and may initiate a signal transduction cascade. EMBO Journal 1996, 15:4919-27.
[21] Wiesmann C, Fuh G, Christinger H W, Eigenbrot C, Wells J A, de Vos A M: Crystal structure at 1.7 A resolution of VEGF in complex with domain 2 of the Flt-1 receptor. Cell 1997, 91:695-704.
[22] Christinger H W, Fuh G, de Vos A M, Wiesmann C: The Crystal Structure of Placental Growth Factor in Complex with Domain 2 of Vascular Endothelial Growth Factor Receptor-1. J Biol Chem 2004, 279:10382-8.
[23] Markovic-Mueller S, Stuttfeld E, Asthana M, Weinert T, Bliven S, Goldie K N, Kisko K, Capitani G, Ballmer-Hofer K: Structure of the Full-length VEGFR-1 Extracellular Domain in Complex with VEGF-A. Structure 2017, 25:341-52.
[24] Ferrara N, Chen H, Davis-Smyth T, Gerber H-P, Nguyen T-N, Peers D, Chisholm V, Hillan K J, Schwall R H: Vascular endothelial growth factor is essential for corpus luteum angiogenesis. Nature Medicine 1998, 4:336-40.
[25] Gerber H P, Vu T H, Ryan A M, Kowalski J, Werb Z, Ferrara N: VEGF couples hypertrophic cartilage remodeling, ossification and angiogenesis during endochondral bone formation. Nature Med 1999, 5:623-8.
[26] Gerber H P, Hillan K J, Ryan A M, Kowalski J, Keller G-A, Rangell L, Wright B D, Radtke F, Aguet M, Ferrara N: VEGF is required for growth and survival in neonatal mice. Development 1999, 126:1149-59.
[27] Gerber H P, Kowalski J, Sherman D, Eberhard D A, Ferrara N: Complete inhibition of rhabdomyosarcoma xenograft growth and neovascularization requires blockade of both tumor and host vascular endothelial growth factor. Cancer Res 2000, 60:6253-8.
[28] de Vries C, Escobedo J A, Ueno H, Houck K, Ferrara N, Williams L T: The fms-like tyrosine kinase, a receptor for vascular endothelial growth factor. Science 1992, 255:989-91.
[29] Sarrazin S, Lamanna W C, Esko J D: Heparan sulfate proteoglycans. Cold Spring Harb Perspect Biol 2011, 3.
[30] Woodard K T, Liang K J, Bennett W C, Samulski R J: Heparan Sulfate Binding Promotes Accumulation of Intravitreally Delivered Adeno-associated Viral Vectors at the Retina for Enhanced Transduction but Weakly Influences Tropism. J Virol 2016, 90:9878-88.
[31] Houck K A, Leung D W, Rowland A M, Winer J, Ferrara N: Dual regulation of vascular endothelial growth factor bioavailability by genetic and proteolytic mechanisms. J. Biol. Chem 1992, 267:26031-7.
[32] Ferrara N: Binding to the extracellular matrix and proteolytic processing: two key mechanisms regulating vascular endothelial growth factor action. Mol Biol Cell 2010 21:687-90.
[33] Malyala P, Singh M: Endotoxin limits in formulations for preclinical research. J Pharm Sci 2008, 97:2041-4.
[34] Bogdanovich S, Kim Y, Mizutani T, Yasuma R, Tudisco L, Cicatiello V, Bastos-Carvalho A, Kerur N, Hirano Y, Baffi J Z, Tarallo V, Li S, Yasuma T, Arpitha P, Fowler B J, Wright C B, Apicella I, Greco A, Brunetti A, Ruvo M, Sandomenico A, Nozaki M, Ijima R, Kaneko H, Ogura Y, Terasaki H, Ambati B K, Leusen J H, Langdon W Y, Clark M R, Armour K L, Bruhns P, Verbeek J S, Gelfand B D, De Falco S, Ambati J: Human IgG1 antibodies suppress angiogenesis in a target-independent manner. Signal Transduct Target Ther 2016, 1.
[35] Saishin Y, Takahashi K, Lima e Silva R, Hylton D, Rudge J S, Wiegand S J, Campochiaro P A: VEGF-TRAP(R1R2) suppresses choroidal neovascularization and VEGF-induced breakdown of the blood-retinal barrier. J Cell Physiol 2003, 195:241-8.
[36] Silva R L E, Kanan Y, Mirando A C, Kim J, Shmueli R B, Lorenc V E, Fortmann S D, Sciamanna J, Pandey N B, Green J J, Popel A S, Campochiaro P A: Tyrosine kinase blocking collagen IV-derived peptide suppresses ocular neovascularization and vascular leakage. Sci Transl Med 2017, 9.
[37] Barleon B, Totzke F, Herzog C, Blanke S, Kremmer E, Siemeister G, Marme D, Martiny-Baron G: Mapping of the sites for ligand binding and receptor dimerization at the extracellular domain of the vascular endothelial growth factor receptor FLT-1. Journal of Biological Chemistry 1997, 272:10382-8.
[38] Roberts C J: Therapeutic protein aggregation: mechanisms, design, and control. Trends Biotechnol 2014, 32:372-80.
[39] Ratanji K D, Derrick J P, Dearman R J, Kimber I: Immunogenicity of therapeutic proteins: influence of aggregation. J Immunotoxicol 2014, 11:99-109.
[40] Wang X, Mathieu M, Brezski R J: IgG Fc engineering to modulate antibody effector functions. Protein Cell 2018, 9:63-73.
[41] Yu L, Wu X, Cheng Z, Lee C V, Lecouter J, Campa C, Fuh G, Lowman H, Ferrara N: Interaction between Bevacizumab and Murine VEGF-A: A Reassessment. Invest Ophthalmol Vis Sci 2008, 49:522-7.
[42] Lambert V, Lecomte J, Hansen S, Blacher S, Gonzalez M L, Struman I, Sounni N E, Rozet E, de Tullio P, Foidart J M, Rakic J M, Noel A: Laser-induced choroidal neovascularization model to study age-related macular degeneration in mice. Nat Protoc 2013, 8:2197-211.
[43] Heier J. S., Kherani S., Desai S., Dugel P., Kaushal S., Cheng S. H., Delacono C., Purvis A., Richards S., Le-Halpere A., et al., Intravitreous injection of AAV2-sFLT01 in patients with advanced neovascular age-related macular degeneration: a phase 1, open-label trial, Lancet 390, 2017, 50-61.
[44] Heier Rakoczy E. P., Lai C. M., Magno A. L., Wikstrom M. E., French M. A., Pierce C. M., Schwartz S. D., Blumenkranz M. S., Chalberg T. W., Degli-Esposti M. A. and Constable I. J., Gene therapy with recombinant adeno-associated vectors for neovascular age-related macular degeneration: 1 year follow-up of a phase 1 randomised clinical trial, Lancet 386, 2015, 2395-2403.

What is claimed is:

1. An anti-VEGF agent comprising a VEGF binding portion operatively linked to a Fc-IgG, wherein the VEGF binding portion comprises at least one VEGF binding domain that is an IgG-like domain 2 of VEGFR-1, and wherein the anti-VEGF agent has a VEGF-stimulated mitogenesis-inhibiting ability greater than aflibercept.

2. The anti-VEGF agent of claim 1, wherein the anti-VEGF agent has a vitreous binding ability greater than aflibercept.

3. The anti-VEGF agent of claim 2, wherein the anti-VEGF agent has a vitreous bound VEGF-stimulated endothelial cell proliferation-inhibiting ability greater than aflibercept.

4. The anti-VEGF agent of claim 3, wherein the agent has an increased half-life in vivo compared to aflibercept.

5. The anti-VEGF agent of claim 4, wherein the VEGF binding portion consists essentially of IgG-like domains 1, 2, and 3 of VEGFR-1 (V₁₋₂₋₃).

6. The anti-VEGF agent of claim 5, wherein the anti-VEGF agent comprises amino acid sequence as defined in SEQ ID NO: 1.

7. The anti-VEGF agent of claim 4, wherein the VEGF binding portion consists essentially of IgG-like domains 2 and 3 of VEGFR-1 (V₂₋₃).

8. The anti-VEGF agent of claim 5, wherein the anti-VEGF agent comprises amino acid sequence as defined in SEQ ID NO: 3.

9. The anti-VEGF agent of claim 4, wherein the VEGF binding portion consists essentially of IgG-like domains 1, 2, 3 and 3 of VEGFR-1 (V₁₋₂₋₃₋₃).

10. The anti-VEGF agent of claim 5, wherein the anti-VEGF agent comprises amino acid sequence as defined in SEQ ID NO: 5.

11. The anti-VEGF agent of claim 4, wherein the VEGF binding portion consists essentially of IgG-like domains 2, 3 and 3 of VEGFR-1 (V₂₋₃₋₃).

12. The anti-VEGF agent of claim 5, wherein the anti-VEGF agent comprises amino acid sequence as defined in SEQ ID NO: 7.

13. A pharmaceutical composition comprising a therapeutically effective amount of an anti-VEGF agent of claim 1, and a pharmaceutically acceptable excipient.

14. A method of treating a VEGF-related disorder in a subject in need comprising administering to the subject a therapeutically effective amount of an anti-VEGF agent of claim 1.

15. The method of claim 14, wherein the anti-VEGF agent is directly injected into the affected tissue or organ.

16. The method of claim 15, wherein the said affected tissue or organ is an eye.

17. The method of claim 14, wherein the VEGF-related disorder is selected from the group consisting of neovascular age related macular degeneration (AMD), choroidal neovascularization, proliferative diabetic retinopathy, diabetic macular edema, a retinal vascular obstruction, an ocular tumor, von Hippel Lindau syndrome, retinopathy of prematurity, polypoid choroidal vasculopathy, a colorectal cancer, a lung cancer, a cervical cancer, an endometrial cancer, ovarian cancer, kidney cancer, a schwannoma, a glioma, and an ependymoma.

18. A method of treating a VEGF-related disorder in a subject in need comprising administering to the subject a therapeutically effective amount of the pharmaceutical composition of claim 13.

19. The method of claim 18, wherein the anti-VEGF agent is directly injected into the affected tissue or organ.

20. The method of claim 20, wherein the said affected tissue or organ is an eye.

21. The method of claim 19, wherein the VEGF related disorder is selected from the group consisting of neovascular age related macular degeneration (AMD), choroidal neovascularization, proliferative diabetic retinopathy, diabetic macular edema, a retinal vascular obstruction, an ocular tumor, von Hippel Lindau syndrome, retinopathy of prematurity, polypoid choroidal vasculopathy, a colorectal cancer, a lung cancer, a cervical cancer, an endometrial cancer, ovarian cancer, kidney cancer, a schwannoma, a glioma, and an ependymoma.

22. An anti-Vascular Endothelial Growth Factor (VEGF) agent comprising a VEGF binding portion operatively linked to a Fc-IgG, wherein the VEGF binding portion comprises an IgG-like domain 2 of VEGFR-1 and an IgG-like domain 4 of VEGFR-1, and wherein the VEGF binding portion does not contain an IgG-like domain 3 of VEGFR-1.

23. The anti-VEGF agent of claim 22, wherein the anti-VEGF agent has a vitreous binding ability greater than aflibercept.

24. The anti-VEGF agent of claim 22, wherein the anti-VEGF agent has an increased half-life in vivo compared to aflibercept.

25. The anti-VEGF agent of claim 22, wherein the IgG-like domain 2 of VEGFR-1 has the sequence of amino acid residues 35-129 of SEQ ID NO: 13.

26. The anti-VEGF agent of claim 22, wherein the IgG-like domain 4 of VEGFR-1 has the sequence of amino acid residues 134-229 of SEQ ID NO: 13.

27. The anti-VEGF agent of claim 22, wherein the VEGF agent comprises amino acid residues 27-456 of SEQ ID NO: 13.

28. A pharmaceutical composition comprising a therapeutically effective amount of an anti-VEGF agent of claim 22 and a pharmaceutically acceptable excipient.

29. A polynucleotide encoding the anti-VEGF agent of claim 22.

30. A method of treating a Vascular Endothelial Growth Factor (VEGF)-related disorder in a subject in need comprising administering to the subject a therapeutically effective amount of an anti-VEGF agent, wherein the anti-VEGF agent comprises a VEGF binding portion operatively linked to a Fc-IgG, wherein the VEGF binding portion comprises an IgG-like domain 2 of VEGFR-1 and an IgG-like domain 4 of VEGFR-1, and wherein the VEGF binding portion does not contain an IgG-like domain 3 of VEGFR-1 of claim 22.

31. The method of claim 30, wherein the anti-VEGF agent is directly injected into an affected tissue or organ.

32. The method of claim 31, wherein the affected tissue or organ is an eye.

33. The method of claim 30, wherein the VEGF-related disorder is selected from the group consisting of neovascular age related macular degeneration (AMD), choroidal neovascularization, proliferative diabetic retinopathy, diabetic macular edema, a retinal vascular obstruction, an ocular tumor, von Hippel Lindau syndrome, retinopathy of prematurity, polypoid choroidal vasculopathy, a colorectal cancer, a lung cancer, a cervical cancer, an endometrial cancer, ovarian cancer, kidney cancer, a schwannoma, a glioma, and an ependymoma.

34. The anti-VEGF agent of claim 30, wherein the anti-VEGF agent has a vitreous binding ability greater than aflibercept.

35. The anti-VEGF agent of claim 30, wherein the anti-VEGF agent has an increased half-life in vivo compared to aflibercept.

36. The anti-VEGF agent of claim 30, wherein the IgG-like domain 2 of VEGFR-1 has the sequence of amino acid residues 35-129 of SEQ ID NO: 13.

37. The anti-VEGF agent of claim 30, wherein the IgG-like domain 4 of VEGFR-1 has the sequence of amino acid residues 134-229 of SEQ ID NO: 13.

38. The anti-VEGF agent of claim 30, wherein the VEGF agent comprises an amino acid sequence having at least 90% identity to amino acid residues 27-456 of SEQ ID NO: 13.

39. The anti-VEGF agent of claim 38, wherein the VEGF agent comprises amino acid residues 27-456 of SEQ ID NO: 13.

40. The anti-VEGF agent of claim 30, wherein the VEGF agent comprises an amino acid sequence having at least 90% identity to amino acid residues 35-456 of SEQ ID NO: 13.

41. The anti-VEGF agent of claim 40, wherein the VEGF agent comprises amino acid residues 35-456 of SEQ ID NO: 13.


SEQUENCE LISTING (14 sequences)

SEQ ID NO: 1 - protein, 558 aa, synthetic construct
  REGION 1..558: note = Description of Artificial Sequence: Syntheticpolypeptide
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1674 bp, synthetic construct
  misc_feature 1..1674: note = Description of Artificial Sequence: Syntheticpolynucleotide
  source 1..1674: mol_type = other DNA
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 3 - protein, 459 aa, synthetic construct
  REGION 1..459: note = Description of Artificial Sequence: Syntheticpolypeptide
MVSYWDTGVLLCALLSCLLLTGSSSGIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1377 bp, synthetic construct
  misc_feature 1..1377: note = Description of Artificial Sequence: Syntheticpolynucleotide
  source 1..1377: mol_type = other DNA
atggtcagctactgggacaccggggtcctgctgtgcgcgctgctcagctgtctgcttctcacaggatctagttcaggtatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 5 - protein, 659 aa, synthetic construct
  REGION 1..659: note = Description of Artificial Sequence: Syntheticpolypeptide
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1977 bp, synthetic construct
  misc_feature 1..1977: note = Description of Artificial Sequence: Syntheticpolynucleotide
  source 1..1977: mol_type = other DNA
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcagtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 7 - protein, 560 aa, synthetic construct
  REGION 1..560: note = Description of Artificial Sequence: Syntheticpolypeptide
MVSYWDTGVLLCALLSCLLLTGSSSGIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1680 bp, synthetic construct
  misc_feature 1..1680: note = Description of Artificial Sequence: Syntheticpolynucleotide
  source 1..1680: mol_type = other DNA
atggtcagctactgggacaccggggtcctgctgtgcgcgctgctcagctgtctgcttctcacaggatctagttcaggtatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcagtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 9 - protein, 655 aa, synthetic construct
  REGION 1..655: note = Description of Artificial Sequence: Syntheticpolypeptide
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1965 bp, synthetic construct
  misc_feature 1..1965: note = Description of Artificial Sequence: Syntheticpolynucleotide
  source 1..1965: mol_type = other DNA
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaacccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 11 - protein, 556 aa, synthetic construct
  REGION 1..556: note = Description of Artificial Sequence: Syntheticpolypeptide
MVSYWDTGVLLCALLSCLLLTGSSSGIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1668 bp, synthetic construct
  misc_feature 1..1668: note = Description of Artificial Sequence: Syntheticpolynucleotide
  source 1..1668: mol_type = other DNA
atggtcagctactgggacaccggggtcctgctgtgcgcgctgctcagctgtctgcttctcacaggatctagttcaggtatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaacccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 13 - protein, 456 aa, synthetic construct
  REGION 1..456: note = Description of Artificial Sequence: Syntheticpolypeptide
MVSYWDTGVLLCALLSCLLLTGSSSGIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1368 bp, synthetic construct
  misc_feature 1..1368: note = Description of Artificial Sequence: Syntheticpolynucleotide
  source 1..1368: mol_type = other DNA
atggtcagctactgggacaccggggtcctgctgtgcgcgctgctcagctgtctgcttctcacaggatctagttcaggtatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtcttcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaacccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa